(19) United States
(12) Patent Application Publication
(10) Pub. No.: US 2025/0275701 A1
(43) Pub. Date: Sep. 4, 2025

(54) SYSTEMS AND METHODS FOR ANALYZING, PRESENTING, AND USING INDICATORS OF MIND-BODY FITNESS BASED ON PHYSIOLOGY AND PERFORMANCE METRICS

(71) Applicant: UNIVERSITY OF MARYLAND, BALTIMORE COUNTY, BALTIMORE, MD (US)

(72) Inventors: Justin Brooks, Dayton, MD (US); Sarah Conklin, Pittsburgh, PA (US)

(21) Appl. No.: 19/068,373

(22) Filed: Mar. 3, 2025

Related U.S. Application Data
(60) Provisional application No. 63/559,962, filed on Mar. 1, 2024.
(60) Provisional application No. 63/752,918, filed on Feb. 3, 2025.

(51) Int. Cl. A61B 5/16; A61B 5/0205; A61B 5/024; A63B 24/00; G16H 10/60; G16H 50/30

(52) U.S. Cl. CPC A61B 5/165; A61B 5/0205; A63B 24/0062; G16H 10/60; G16H 50/30; A61B 5/02405

(57) ABSTRACT

Systems and methods for analyzing, presenting, and using indicators of mind-body fitness based on physiology and performance metrics are disclosed. According to an aspect, a system includes an input interface configured to receive one or more acquired performance metrics that are indicative of a person's performance of a predetermined activity. The system also includes cognitive stress analyzer configured to receive one or more acquired physiology metrics that are indicative of a condition of the person. The cognitive stress analyzer is configured to analyze the one or more acquired physiology metrics and the one or more acquired performance metrics to generate an indicator of a mind-body fitness of the person. Further, the system includes a user interface configured to present the indicator of the mind-body fitness of the person.

### CROSS REFERENCE TO RELATED APPLICATIONS

This application claims priority to U.S. Patent Application No. 63/752,918, filed Feb. 3, 2025, and titled COGNITIVE STRESS SCORE, TREATMENT, AND MONITORING, and U.S. Patent Application No. 63/559,962, filed Mar. 1, 2024, and titled COGNITIVE STRESS SCORE, TREATMENT, AND MONITORING; the contents of which are incorporated herein by reference in their entireties.

### TECHNICAL FIELD

The presently disclosed subject matter relates generally to cognitive stress assessment. Particularly, the presently disclosed subject matter relates to systems and method for analyzing, presenting, and using indicators of mind-body fitness based on physiology and performance metrics.

### BACKGROUND

Individual differences in the reflexive physiological reactivity to everyday challenges, perceived threats, and stressors contribute to disease risk and resilience. Acutely, enhanced stress reactions may lead to symptom reports of fatigue, anxiety, or stress yet still adaptively benefit performance. In the long term, exaggerated reactions, whether in magnitude or duration, compromise performance, and increase the subsequent risk for high blood pressure, burnout, heart disease, and other indices of poor health. In laboratory research, exaggerated reactions to the paced serial addition test (PASAT), a cognitive challenge test used to elicit reflexive physiological reactions, predicted 16-year CHD mortality.

Measurement of stress is a challenge and has been approached by a variety of methods. In laboratory settings, paradigms such as the Trier Social Stress Task, the cold pressor test, and the PASAT are used to induce social, physical, or cognitive stress, respectively. Physiological responses to these laboratory stress-inducing tasks are typically recorded and analyzed in phases (baseline, stress exposure, recovery). Both exaggerated and blunted reactions to laboratory stress have been related to psychopathology, cardiovascular morbidity and mortality, traumatic experiences, patterns of health behavior, and social and demographic variables.

In medical settings, serum cortisol is commonly used as a purely physiological measure of stress. Cortisol is often assessed as an awakening response with the area under the curve calculated across several morning measurements. Finally, in the wild, wearable devices use proprietary algorithms on photoplethysmography sensor data to estimate stress load or periods of stress. Although these different methods provide valuable snapshots of stress and are meaningfully associated with risky health outcomes, they do not capture how a person performs under stress, how they appraise the challenge, and how their physiology meets the challenge with reflexive responses facilitated in part by the autonomic nervous system (ANS).

It is well established that cognitive demand induces a reflexive physiologic response (e.g. change in heart rate) via the ANS, providing a causal mechanism that relates rapid decision-making, frequent task switching, and continuous shifts in attention (ubiquitous in the modern workplace and home) to fundamental changes in physiology. Individual differences in physiological responses to cognitive challenges and stressors are crucial in psychological research because they help explain why people react differently to similar situations. Genetic, psychological, and sociocultural factors can influence these differences. For example, some individuals may have a heightened stress response due to genetic predispositions, leading to increased magnitude and or duration of physiological reactions (blood pressure, cortisol, heart rate dynamics, skin conductance). Others might exhibit more resilient physiological responses, which can buffer against the negative effects of stress. These variations can impact mental health outcomes, such as susceptibility to anxiety, depression, and other stress-related disorders.

Despite advancements in neuroimaging technologies, the gold standard assessment of psychological experience remains self-reports, typically gathered through structured or semi-structured interviews, questionnaires, or state probes. However, there is a continuing need for improved systems and techniques for assessing an individual's ability to perform under cognitive stress.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 is a block diagram of a system for analyzing, presenting, and using indicators of mind-body fitness based on physiology and performance metrics in accordance with embodiments of the present disclosure;

FIG. 2 is a flow diagram of a method for analyzing, presenting, and using indicators of mind-body fitness based on physiology and performance metrics in accordance with embodiments of the present disclosure in accordance with embodiments of the present disclosure;

FIG. 3 shows graphs depicting distribution of Yerkes-Dodson Manifold (YDM), performance, and physiology data;

FIG. 4 shows a graph of the coefficient of variation (CV) analysis for YDM, performance, and physiology;

FIG. 5 is a graph depicting resilience-feature correlations of YDM, performance, and physiology;

FIG. 6 is a graph depicting correlation analysis: YDM on the x-axis and resilience on the y-axis;

FIG. 7 is a diagram depicting an overview of the phases of Baseline and Testing in accordance with embodiments of the present disclosure;

FIG. 8 shows graphs depicting the variability among participants for task accuracy, workload, and each of the state probes;

FIG. 9 is a graph depicting standardized accuracy and workload metrics plotted with points colored according to their assigned clusters;

FIG. 10 are graphs depicting cluster analysis of all data points;

FIG. 11 shows graphs depicting the results of cluster analysis using K-Medoids algorithm, highlighting the evaluation of the optimal number of clusters;

FIG. 12 is an example report of generated analysis information of a person about how the person's body responds to stress by analyzing the person's heart response;

FIG. 13 is an example report of task switching analysis information;

FIG. 14 is an example report of enumeration data;

FIG. 15 is an example report of spatial working memory data;

FIG. 16 is an example heart activity report;

FIG. 17 is an example heart rate variability report;

FIG. 18 is an example report showing average workload for each session;

FIG. 19 is a graph shows plots of how stressful and demanding a person rated difficulty levels across tasks;

FIG. 20 is a graph showing average response as compared to input of two questions;

FIG. 21 is a graph depicting a plot of the difference between an estimation of task success and a person's actual accuracy for each level; and

FIG. 22 is a graph depicting a plot of the difference between an estimation of task success and actual accuracy for each task.

### SUMMARY

The presently disclosed subject matter relates to systems and methods for analyzing, presenting, and using indicators of mind-body fitness based on physiology and performance metrics. According to an aspect, a system includes an input interface configured to receive one or more acquired performance metrics that are indicative of a person's performance of a predetermined activity. The system also includes cognitive stress analyzer configured to receive one or more acquired physiology metrics that are indicative of a condition of the person. The cognitive stress analyzer is configured to analyze the one or more acquired physiology metrics and the one or more acquired performance metrics to generate an indicator of a mind-body fitness of the person. Further, the system includes a user interface configured to present the indicator of the mind-body fitness of the person.

### DETAILED DESCRIPTION

The following detailed description is made with reference to the figures. Exemplary embodiments are described to illustrate the disclosure, not to limit its scope, which is defined by the claims. Those of ordinary skill in the art will recognize a number of equivalent variations in the description that follows.

Articles “a” and “an” are used herein to refer to one or to more than one (i.e. at least one) of the grammatical object of the article. By way of example, “an element” means at least one element and can include more than one element.

“About” is used to provide flexibility to a numerical endpoint by providing that a given value may be “slightly above” or “slightly below” the endpoint without affecting the desired result.

The use herein of the terms “including,” “comprising,” or “having,” and variations thereof is meant to encompass the elements listed thereafter and equivalents thereof as well as additional elements. Embodiments recited as “including,” “comprising,” or “having” certain elements are also contemplated as “consisting essentially of” and “consisting” of those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a range is stated as between 1%-50%, it is intended that values such as between 2%-40%, 10%-30%, or 1%-3%, etc. are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

Embodiments of the present disclosure provide integrative frameworks that present an indicator of a mind-body fitness of a person with objective measurements of physiological and activity (or behavioral) function. Studies have shown that healthy individuals and those with psychiatric conditions-such as depression, insomnia, schizophrenia, and fibromyalgia-often exhibit significant discrepancy between their subjective reports and objective assessments. For example, in insomnia sleep-wake state discrepancy is a common phenomenon where patients report frustratingly long sleep onset latency or duration in their sleep diary entries or interviews, however, on polysomnography their sleep metrics are typical. Conversely, in other work, discordant, positively biased perceptions were linked to more positive outcomes, such as reduced morbidity and mortality.

It is noted that the concept of “biotypes” can provide a more global assessment of functional type, capturing the interplay between cognitive, physiological, and/or psychological processes. This framework can be applied to inform precision medicine approaches to treatment. For example, in psychiatry, biotypes can be used to identify subgroups within conditions like depression, addiction, insomnia, and schizophrenia, improving the prediction of treatment outcomes. This integrative approach holds the potential to undercover meaningful patterns that reveal shared characteristics in terms of perception, cognition, and physiological responding, bridging the gap between what individuals feel and what their physiological and behavioral patterns reveal. By identifying these patterns, the biotype framework can offer a powerful tool.

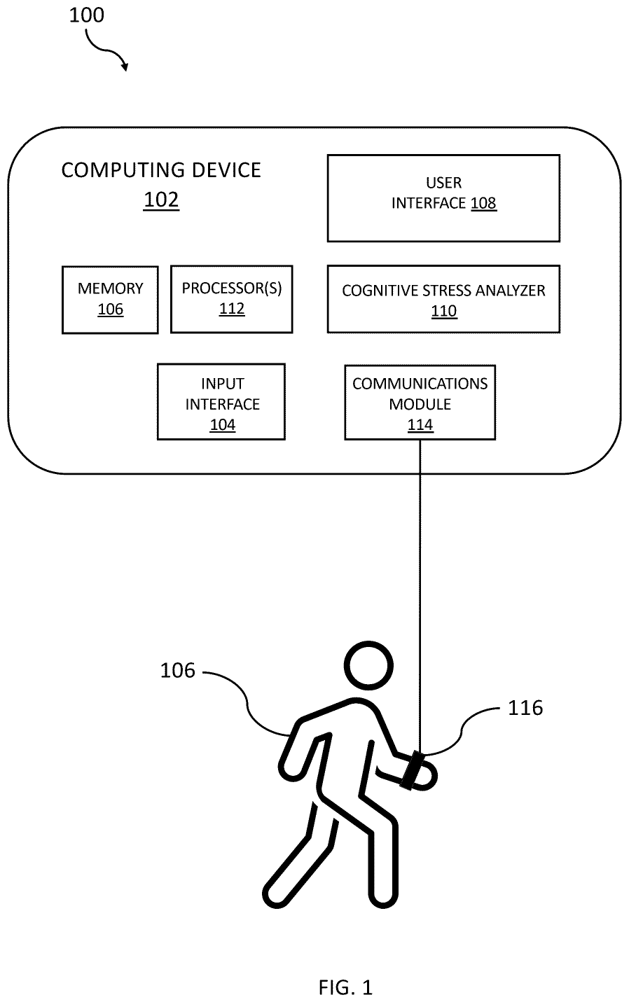

FIG. 1 illustrates a block diagram of a system 100 for analyzing, presenting, and using indicators of mind-body fitness based on physiology and performance metrics in accordance with embodiments of the present disclosure. Referring to FIG. 1, the system 100 includes a computing device 102 having an input interface 104 configured to receive one or more acquired performance metrics that are indicative of a person's performance of a predetermined activity. For example, the input interface 104 can receive data indicative of a performance metric such as an accuracy of a person 106 discharging a firearm at a practice target. Examples of acquired performance metrics include, but are not limited to, firearm accuracy activity, cognitive testing, computing device use activity, race car driving, eSports performance, and the like. In an example, the data can be acquired by another device and input to the computing device 102 via the input interface 104. The received data can be stored in memory 106. In another example, the data can indicate a metric of computing device use activity, such as the person's 106 entry of inputs into a keyboard, touchscreen display, a mouse, or other device of a user interface 108. This acquired data can indicate the person's 106 ability to perform a physical activity. For example, some acquired data may indicate that one person is better at another person at performing the physical activity.

The computing device 102 includes a cognitive stress analyzer 110. Functionalities of a cognitive stress analyzer 110 as described herein can be implemented by suitable hardware, software, and/or firmware. For example, the cognitive stress analyzer 110 can be implemented by one or more processors 112 implementing instructions stored in memory 106. In this example, the cognitive stress analyzer 110 resides within the computing device 102. Alternatively, for example, the functionalities of the cognitive stress analyzer 110 may be implemented at another computing device and the computing device 102 may communicate with the other computing device with a communications module 114 for utilizing the cognitive stress analyzer.

The cognitive stress analyzer 110 is configured to receive one or more acquired physiology metrics that are indicative of a condition of the person 106. For example, the person 106 can wear a wearable device 116 configured to acquire physiology metrics, such as heart rate. In this example, the person 106 is wearing a smart watch configured to acquire a heart rate of the person 106 and to communicate the acquired heart rate data to the computing device 102. As an alternative, for example, the physiology metrics may be acquired by another such as a chest monitoring device for detecting heart dynamics. The wearable device 116 device can wirelessly communicate the acquired heart rate data to the computing device where the data is received by the communications module 114 and stored in memory 106. Although only one wearable device (e.g., wearable device 116) is described in this example as acquiring physiology metrics, it should be appreciated that any suitable number of physiology metrics of any type can be acquired by any suitable number of devices

The cognitive stress analyzer 110 is configured to analyze the acquired physiology metric(s) and the acquired performance metric(s) to generate an indicator of a mind-body fitness of the person 106. For example, the cognitive stress analyzer 110 can retrieve the acquired physiology metrics and the acquired performance metrics stored in memory 106 for generating an indicator of mind-body fitness of the person 106. In an example of generating the indicator of mind-body fitness, the cognitive stress analyzer 110 can derive a measure of a Yerkes-Dodson Manifold (YDM) based on a ratio of a z-scored performance of the person 106 at an activity and a z-scored physiology metric of the person 106. [By measuring physiological workload (i.e., heart rate variability in the ECG signal) across multiple domains of cognition and levels of task demand, the system measures mind: body fitness as a single coordinate in the multidimensional space of normalized cognitive performance and normalized physiological workload. Our clustering approach identifies phenotypes of cognitive stress responsivity and responsivity.]

The user interface 108 is configured to present the indicator of a mind-body fitness of the person 106. For example, the cognitive stress analyzer 110 can control a display of the user interface 108 to display the indicator of the mind-body fitness of the person 106. The indicator may be presented as a number, a graphic, or other suitable means for presenting the indicator. Alternatively, for example, the indicator may be presented audibly such as by a speaker of the user interface 108.

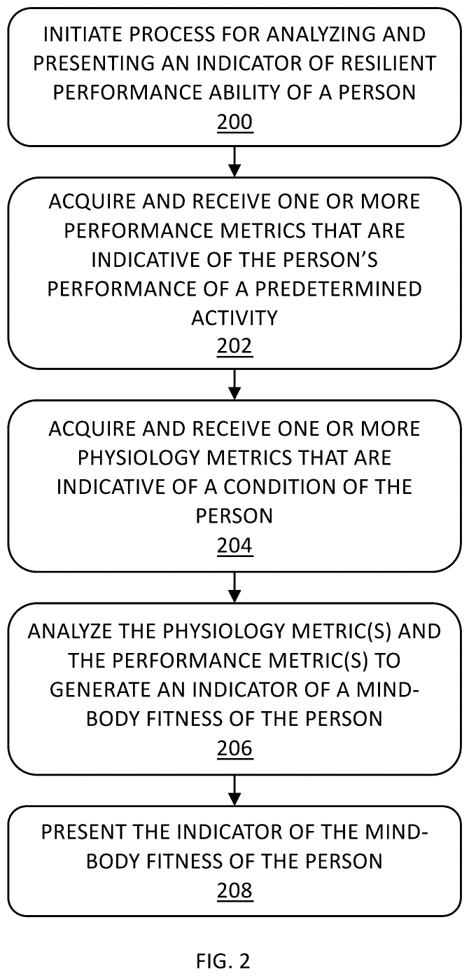

FIG. 2 illustrates a flow diagram of a method for analyzing, presenting, and using indicators of mind-body fitness based on physiology and performance metrics in accordance with embodiments of the present disclosure in accordance with embodiments of the present disclosure. The method of FIG. 2 is described by example as being implemented by the system 100 shown in FIG. 1, but it should be appreciated that the method may alternatively be implemented by any other system or by one or more computing devices.

The method of FIG. 2 includes initiating 200 process for analyzing and presenting an indicator of mind-body fitness. For example, the person 106 shown in FIG. 1 or another user of the computing device 102 may interact with the user interface 108 to initiate a process for analyzing and presenting an indicator of mind-body fitness the person 106. As an example, the cognitive stress analyzer 110 can control a display of the user interface 108 to display an interface for interaction to initiate the process. The person 106 or another can use the user interface 108 (e.g., a keyboard, mouse or touchscreen display) to identify the person 106 being tested for mind-body fitness, to specify the wearable device 116 for acquiring a physiology metric of the person 106, and to specify a performance test to be administered to the person 106.

The method of FIG. 2 includes acquiring and receiving 202 one or more performance metrics that are indicative of a person's performance of a predetermined activity. Continuing the aforementioned example, the person 106 can begin and complete a physical activity that is a test of their performance of the activity. For example, the person 106 can complete a firearm test for accuracy at shooting at a target. The input interface 104 can receive a performance metric of the results of the firearm test. For example, the performance metric can indicate the results of the person hitting within different predefined areas of a target. This can be detected by a camera and then input into the input interface 104. Alternatively, a keyboard or other suitable user interface can input the performance metric into the input interface 104.

The method of FIG. 2 includes acquiring and receiving 204 one or more physiology metrics that are indicative of a condition of the person. Continuing the aforementioned example, the person 106 is wearing a smart watch configured to acquire a heart rate of the person 106 and to communicate the acquired heart rate data to the computing device 102 for storage in memory 106 where it is accessible by the cognitive stress analyzer 110. The physiology metric(s) can be acquired within a time period of the one or more performance metrics being acquired. It is noted that one or more physiology metrics can be acquired and used for generating the indicator of resilient performance. Example physiology metrics include, but are not limited to, heart rate variability, blood pressure, oxygen saturation, respiratory rate, body temperature, blood glucose level, oxygen consumption, electrodermal activity, electroencephalography, pupillometry, gaze position and velocity, muscle activation, and the like.

The method of FIG. 2 includes analyzing 206 the physiology metric(s) and the performance metric(s) to generate an indicator of a mind-body fitness of the person. Continuing the aforementioned example, the cognitive stress analyzer 110 can analyze the acquired physiology metric(s) and the acquired performance metric(s) to generate an indicator of a mind-body fitness of the person 106. For example, the cognitive stress analyzer 110 can retrieve the acquired physiology metrics and the acquired performance metrics stored in memory 106 for generating an indicator of mind-body fitness of the person 106. In an example of generating the indicator of mind-body fitness, the cognitive stress analyzer 110 can derive a measure of a Yerkes-Dodson Manifold (YDM) based on a ratio of a z-scored performance of the person 106 at an activity and a z-scored physiology metric of the person 106.

The method of FIG. 2 includes presenting 208 the indicator of a mind-body fitness of the person. Continuing the aforementioned example, the cognitive stress analyzer 110 can control a display of the user interface 108 to display the indicator of the mind-body fitness of the person 106. The indicator may be presented as a number, a graphic, or other suitable means for presenting the indicator.

In embodiments, the cognitive stress analyzer 108 can receive indicators of mind-body fitness of multiple other people. Further, the cognitive stress analyzer 108 can analyze the indicator of the mind-body fitness of the person 106 in comparison to the indicators of mind-body fitness of the other people. Further, the cognitive stress analyzer 108 can analyze the indicator of the mind-body fitness of the person 106 in comparison to the indicators of mind-body fitness of a plurality of other people. The cognitive stress analyzer 108 can also use the user interface 108 to present the analysis.

Embodiments of the present disclosure can be utilized to generate and present an indicator of a mind-body fitness of a person. As referred to herein, the term “mind-body fitness” relates to a person's resilience, performance under pressure, ability to handle stress, and the like. For example, mind-body fitness can relate to the ability of a person to maintain or recover optimal functioning in the face of significant stressors, as an individual or among a team, and/or over prolonged duration. In an example, a mind-body fitness can be relevant for military personnel. Other example mind-body fitness characteristics of a person include, but are not limited to, stress tolerance, anxiety regulation, burnout resistance, mental stamina, cognitive endurance, psychological resilience, adaptive capacity, performance resilience, neural efficiency, focused adaptability, emotional regulation, executive function under load, cognitive load tolerance, physiological adaptability, mental toughness, decision-making ability under stress, and the like. Since military personnel often encounter challenging and demanding situations that can affect the capacity for cognitive or mind-body fitness, and subsequently provide individualized or team training to promote resilience remains a top priority for developing other military personnel. Systems and methods according to present disclosure utilize physiology metrics and performance metrics for determining mind-body fitness and also for determining cardiovascular disease risk, anxiety, hardiness, grit, vigilance, and various other human traits.

Heart rate variability (HRV) is a measure of the variation in time intervals between consecutive heartbeats. Broadly speaking HRV reflects the balance and flexibility of the sympathetic and parasympathetic branches of the autonomic nervous system and has been related to diverse indices of health, fitness, stress, and mortality risk. HRV can be computed in both time and frequency domains that capture different aspects of heart rate dynamics. The root mean square of successive differences (RMSSD) between adjacent R-R intervals in the electrocardiogram (ECG) signal reflects the high-frequency component of HRV, which is influenced primarily by the parasympathetic nervous system. Within and beyond the Military context, HRV can be utilized as an index of the activation of the autonomic nervous system. Higher indices of HRV, indicate greater challenge. Lower HRV, on the other hand, has been linked to psychological stress, post-traumatic stress disorder, and impaired performance, as well as a number of poor indicators of health.

With respect to resilience, use of these tools has two implications that inform the hypotheses for applications in embodiments of the present disclosure. First, because of the non-linear relationship between performance and physiology, there is likely greater information (more inter-individual variance) in a combined assessment of physiology and performance than in either metric independently. This was examined by comparing the coefficient of variation (CV) of 1) physiological assessment, 2) performance assessment, and 3) a metric that combines performance and physiology. An example of this is referred to as the Yerkes Dodson Manifold (YDM). The CV is a statistical approach used to gauge the relative dispersion of data points in a series around the mean. It is defined as the ratio of the standard deviation to the mean. In embodiments, the cognitive stress analyzer 110 is configured to compare a coefficient of variation of one or more acquired physiology metrics and one or more acquired performance metrics of the person 108 to generate an indicator of a mind-body fitness of the person.

In example applications, the indicator (or metric) of mind-body fitness of a person can be used in a military setting as a screening tool and training target for resilient performance. It can be expected that for a given level of performance, relatively less physiological arousal can be tied to great resiliency. To address the nature of a screening metric, it may be considered whether a combination metric of physiology and performance provide more information across individuals than either in alone or in isolation. In hypothesis, it was considered whether the combined metric would show a great CV than performance or physiology. Another hypothesis was that an individual who exhibits a greater degree of adaptive physiological activity (more variability in heart rate) for a given level of performance can be more likely to be resilient (perform at a consistent level over time) than those who maintain the same level of performance with less physiological variability in heart rate. Evidence for this hypothesis may be stronger relationship between RP and the YDM than the association between resilient performance (RP) and performance or physiology independently.

In experiments for firearm target practice, participants were volunteers (N=30, 17 males, mean age=25.28, SD=2.91) recruited for study on neuro-feedback and performance under stress. The stressful shooting simulation was conducted in virtual reality (VR) which was set as a 9-station shooting range in the desert. In an example, a VR device can interface with the input interface 104 shown in FIG. 1 for receiving acquired performance metric data that is indicative of a person's performance of target shooting. In the study, participants completed a friend-foe discrimination challenge where they had to shoot enemy targets (in red with rifles held up aiming toward participants) and refrain from shooting friendly targets (in blue with rifles held down at the side aiming toward the ground). The shooting simulation was scripted and completed 6 times, during the daytime, over several days while 2-lead ECG was continuously recorded as physiology metric acquisition. Task difficulty was manipulated by target exposure time and included two levels (low, high) which were individualized to each participant based on a thresholding session used to titrate difficulty. All participants first completed a low-difficulty baseline session. In the high-difficulty condition, targets were presented very briefly, forcing participants to make rapid decisions on whether to shoot or not. In the low-difficulty condition, the target exposure time was longer, permitting easier discrimination between friendly and enemy targets. For each participant, low difficulty was determined as the target exposure time required for 90% shooting accuracy, whereas high difficulty was determined as the target exposure time that produced 50% accuracy for shots on enemy targets. For each condition, 90% of targets were enemies and 10 were friends. Each block contained 90 shooting targets for a total of 720 trials per session.

In the aforementioned study, marksmanship performance was evaluated using response time (RT) for hits only. Resilient performance (RP) was quantified by measuring adaptation to high-difficulty blocks of trials, spanning approximately 20 minutes, where more consistency and decreasing response times demonstrated better adaptation, and resilience, to difficulty. Conversely, less resilient performance was characterized as more variable response time during high-difficulty blocks, reflecting less adaptability.

ECG was recorded with lead II configuration using disposable electrodes and the Biosemi ActiveTwo system. The ECG was assessed with the time domain measure of HRV, the root mean square of successive differences (RMSSD) between normal heartbeats. The ECG signal was pre-processed in Python using the Biosppy toolbox. Performance and physiology data were synchronized using Lab Streaming Layer. Other physiological parameters may be collected and utilized.

To derive a measure of the Yerkes-Dodson Manifold in this example, a ratio of the z-scored performance (response time for enemy hits only) and the z-scored physiology (RMSSD) were derived. Two analyses were conducted. First, the CV was examined across the performance measures and physiology as an assessment of the information contained within each metric. Second, to examine the utility of these parameters as screen assessments, each metric was computed at baseline. During the baseline, the participants completed a series of high-stress trials where RP was examined. Finally, the Pearson correlation coefficient between the RP and each metric was computed: performance at baseline, physiology at baseline, and the YDM at baseline.

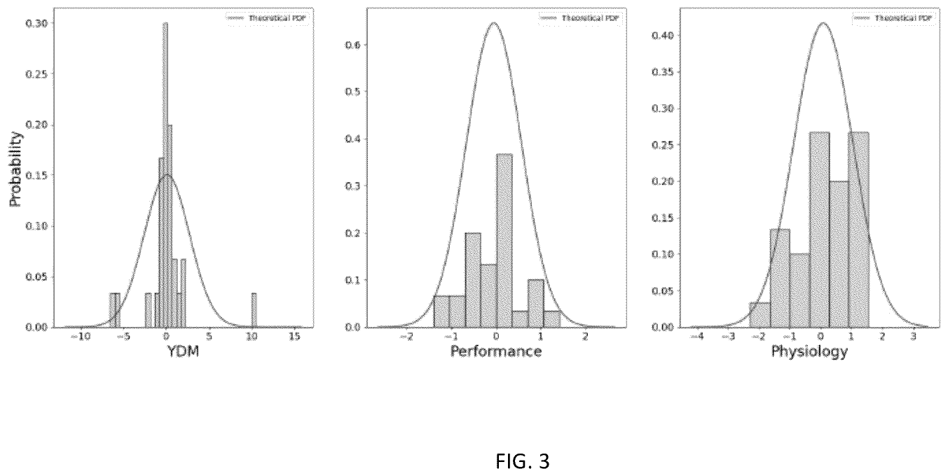

Regarding results of the study, a preliminary analysis focused on assessing the distribution of the performance, physiology, and YDM data. FIG. 3 illustrates graphs depicting distribution of YDM, performance, and physiology data. This figure shows the distributions of YDM, performance, and physiology data after applying the Yeo-Johnson transformation to YDM. The transformation was utilized to approximate a normal distribution, enhancing the robustness and interpretability of the analysis. It is noted that YDM=Yerkes Dodson Manifold, performance=response time for hits only; physiology=RMSSD=Root mean square of successive differences as a measure of heart rate variability; and Theoretical PDF=Theoretical Probability Density Function.

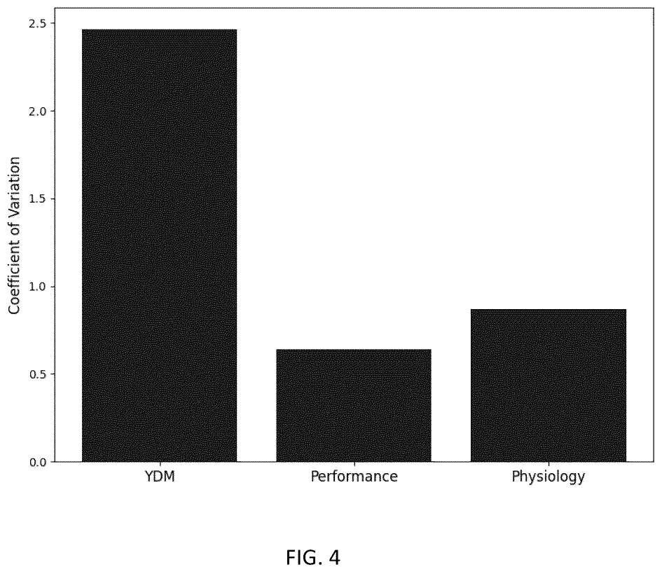

To test a first hypothesis, the CV was examined for performance, physiology, and the YDM. It was hypothesized that the CV for the YDM ratio would be higher than that for performance or physiology alone. As shown in FIG. 3, the mean values were originally centered around zero. To facilitate the CV analysis, a constant value (1) was added to each feature, shifting their means away from zero. This adjustment enhanced the suitability of the data for CV analysis. FIG. 4 depicts a graph of CV analysis for YDM, performance, and physiology. This figure presents the calculated CV values for YDM, performance, and physiology data. Each feature's mean was shifted away from zero to facilitate the CV analysis. It is noted that YDM=Yerkes Dodson Manifold, performance=response time for hits only; and physiology=RMSSD=Root mean square of successive differences as a measure of heart rate variability. As can be seen in FIG. 4, the CV for YDM=2.46 was greater than the CV for performance=0.64, or the CV for physiology=0.87. These CV values provide insights into the variability of each feature and support a hypothesis regarding the YDM ratio's higher CV compared to performance accuracy (Response Time for Hits only) and physiology (RMSSD).

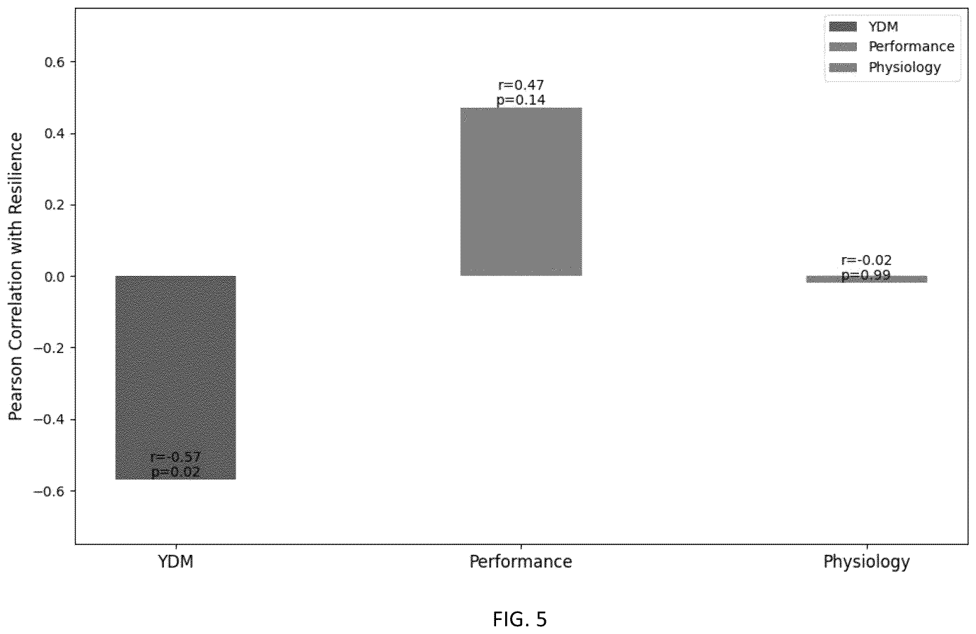

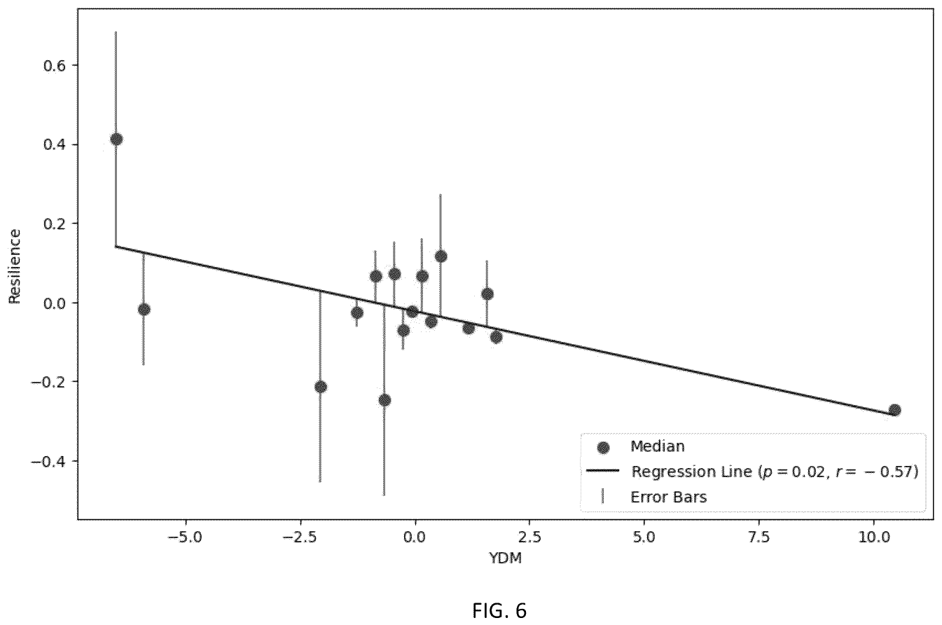

Another analysis examined the correlation between the RP and each of the following metrics at baseline only: YDM metric, performance alone, and physiology alone. The hypothesis was that YDM would show a greater correlation with RP than the two metrics alone. Given the small sample size and sensitivity to outliers, a binning approach was adopted by dividing the x-axis into constant bins (0.2) and calculated the median resilience value within each bin. FIG. 5 is a graph depicting resilience-feature correlations of YDM, performance, and physiology. The YDM was significantly correlated with resilient performance, whereas the association between resilient performance and physiology or performance alone was not significant. It is notes that YDM=Yerkes Dodson Manifold, performance=response time for hits only; physiology=RMSSD=root mean square of successive differences as a measure of heart rate variability. Further, FIG. 6 is a graph depicting correlation analysis: YDM on the x-axis and resilience on the y-axis. Error bars represent the standard deviation of resilience within each YDM bin, with a bin size of 0.2. The correlation between RP and performance was 0.47 (p=0.14). For RP and physiology, the correlation was −0.02 (p=0.99). In contrast, the YDM showed a statistically significant correlation of −0.57 (p=0.02) indicating lower YDM during the baseline was associated with adaptation to high-difficulty blocks by showing decreased RT for shooting targets (Hits only). Without the binning procedure, using a standard, parametric Pearson correlation on the continuous data, the results were similar: the correlation between RP and performance alone was r=0.13 (p=0.50), physiology alone was r=0.02 (p=0.90), and the YDM was r=−0.26 (p=4c-4).

The outcome of CV analysis revealed that the YDM contained more information (higher coefficient of variation) than physiology or performance independently. This can be a significant value when developing a screening metric for military tasks, positions, and operations. The second analysis between RP and YDM validates that physiology-normalized performance provides a useful measure that predicts resilient performance over time. The directionality of the relationship was negative such that lower YDM (Response Time for Hits/RMSSD HRV) at baseline was associated with increased RP defined as an adaptation to high-difficulty shooting blocks revealed by improved RTs.

Experiments were conducted for assessing cognitive performance and physiological workload to test for distinct biotypes based on performance accuracy and the associated workload. It was shown that these groups correlate with subject-objective discrepancies in success and workload, with the highest-performing biotype showing the least discrepancy. In a study, participants were tasked with completing a brief battery (<1 hour) of increasingly challenging cognitive tasks while seated at a computer. A real-time workload algorithm simultaneously processed ECG to classify the state as in or not in workload on a millisecond by millisecond basis. Individual variability was analyzed using a clustering approach that incorporated standardized performance accuracy and standardized workload during the tasks.

In the study, evidence was found for 3 distinct, tightly-formed clusters: (i) the highest performers with the relatively lowest workload, (ii) average to high performers with a relatively high workload, and (iii) average to low performers with a range of workload. Participants in the highest and lowest performing cluster showed the least discrepancy between their appraisal of their temporal and mental demand, compared to corresponding objective indices but the opposite was true for success. Participants in the highest performing cluster showed the greatest discrepancy between their appraisal of success compared to their accuracy on the tasks suggesting that they underestimated their task accuracy.

In this study, participants completed a 60-minute laboratory session. First, brief study questionnaires (demographic information and questions about video game history) were completed on a desktop computer. Following this, participants were instructed to remain seated, at the same computer, for a 5-minute, baseline recording.

Following the baseline period, participants completed three cognitive tasks (<15 minutes each) on the same computer, in random order, while cardiovascular and eye activity were recorded. For the first round of each cognitive task, there was a practice round that could be repeated to ensure each participant understood the task. Each cognitive task was presented as three blocks varying in difficulty (easy, medium, and hard). In between each block, for each task, participants completed state probes that asked them to separately indicate how challenging, successful, stressful, mentally demanding, and hurried the prior block felt to the participant. After they completed the state probes, the next block began until all tasks and all blocks were complete. Participants completed nine total blocks, three for each task, in randomized order.

To collect resting baseline data, 5 minutes was recorded while the participant watched a relaxing video of a lava lamp. Before the recording, participants were instructed to adhere three adhesive ECG electrodes: one below the clavicle near the right shoulder, one below the clavicle near the left shoulder, and one on the lower left abdomen. Participants were asked to keep both feet on the floor and remain seated.

The cognitive tasks were developed from an open-source cognitive battery. The battery was adapted into an abbreviated set integrated with an ECG sensor and the code expanded so that task difficulty was manipulated into blocks of easy, medium, and hard trials. The ECG signal was ingested by a machine learning algorithm that estimates cognitive workload from ECG data and predicts NASA TLX workload and temporal demand. Completion of each of the three tasks generated a raw accuracy score for each level of task difficulty (easy, medium, and hard for Tasks 1, 2, and 3). Challenge Reactivity groups were generated for overall performance that characterizes the physiologic cost of performance on the cognitive battery across all tasks and difficulty levels. Proportional variation in gender and race across Challenge reactivity groups were examined with chi-square analysis computed using an online calculator.

Enumeration measures counting ability. Trials began with a fixation cross in the center of the screen. Participants were instructed to look at the center of the screen, count the number of circles (varied between 5 and 9), and answer using a mouse click. Difficulty in the task was manipulated by the time participants had to count the circles. Each condition (easy, medium, and hard) was tested 20 times.

TS measures the flexibility of selective attention and shifting attention between different goals. In this task, participants were asked to indicate if the targe digit was odd/even or if the target digit was higher/lower than five as a function of the color of the task cue. Accuracy for switch and non-switch trials was calculated as a switch cost. The switch cost was determined by the difference in reaction time between switch and non-switch trials. Digits 1-4, and 6-9 were used for target stimuli. Tasks changed as a function of the color and shape of the task cue. When task cues were red and square, participants answered whether a target digit was odd/even by pressing a specific key. When cues were blue and diamond-shaped, a different key was pressed. Each participant completed 30 trials for each stimulus condition. Task difficulty was manipulated by modifying the reaction time deadline, structured in a block design format, and characterized as easy, medium, and hard.

The task assesses visual-spatial short-term memory capacity. On each trial, 1 of 16 squares is colored red briefly (other squares are gray). A new grid of non-colored squares is shown and participants are asked to “replay” the pattern or red squares by clicking on the squares colored red in order on the prior display. To manipulate the difficulty of this task, the number of squares increased by 4, 6, and 8 for easy, medium, and hard difficulty, respectively.

To capture individual differences in state appraisal during the tasks, at the end of each block, the screen paused, and the participant was prompted to complete several brief questions about their level of perceived stress and frustration, challenge and effort, mental demand, and pace that best represented their state during the block immediately preceding the prompt. The response option for each state probe was a Likert scale of 0-20 with 0 conveying no stress, challenge, or workload and 20 conveying the highest level. State probe responses were used to generate metrics of subject-objective discrepancy/perceptual bias to test the second hypothesis.

To test the hypothesis that cluster would show less discrepancy on subject-objective assessments during tasking, the following metrics were generated using the difference between normalized (subjective) state probe responses and the corresponding (more objective) measure for three comparisons including 1) the success discrepancy metric was the state success probe minus objective task accuracy, 2) the workload discrepancy metric was calculated as the subject workload state probe workload from the ECG algorithm, and 3) the temporal demand discrepancy metric included the temporal demand probe minus workload from the algorithm. Positive values indicate over-estimation. Negative values indicate under-estimation.

Heat activity was recorded using a custom-built ECG device. The device includes an Arduino box connected to a set of wired electrodes with three leads. Each lead was attached to a disposable sticker placed below the left and right clavicles (near the shoulders) and on the lower left abdomen. The box was installed under the table, allowing participants to manually connect the electrode wires after placing the adhesive stickers. This setup provided a secure, minimal invasive, and reliable connection for data collection.

The ECG signals were collected using a 3-channel configuration, with a sampling rate of 250 Hz. The total collected data was about 62.5 hours. Data was segmented into 30-second samples with a 1-second increment between each sample. During preprocessing, the data was filtered and the signal-to-noise ratio (SNR) was calculated for each sample. Samples with an SNR below 4 dB were classified as invalid and removed. Approximately 3.5% of the data was removed as noise.

To calculate workload, the heart rate was computed for each 30-second sample. Standardization was applied for each subject to minimize subject variability. Workload was calculated during baseline and each of the three cognitive tasks. Subsequently, the workload change was computed from the baseline by subtracting the baseline workload from the workload for each of the three tasks. The final workloads were standardized with the following equation:

w=z(M(zᵢ(x_{i,tasks}))−M(zᵢ(x_{i,baseline})))

where, w was the final workload, x_{i,tasks} represented the heart rate for three cognitive tasks for subject i, and x_{i,baseline} was the baseline heart rate for subject i only. zᵢ denoted standardization using data only from subject i. z represented standardization across all data. M was the workload model prediction.

In some studies, workload models were developed to detect cognitive resource depletion using physiological features extracted from tasks such as mental arithmetic, simulated navigation and decision-making, and visuospatial/sensorimotor processing in another study. The models demonstrated strong generalizability across diverse tasks and participant samples. Two versions of the model were evaluated: one integrating both EEG and ECG data and another relying solely on ECG data. For this study, the ECG-only model was selected for workload predictions due to its practicality, case of data collection, and reliable performance in accurately detecting cognitive depletion.

The model architecture consists of two fully connected hidden layers, each with 20 nodes, designed to capture meaningful representations of ECG features for binary workload classification. To prevent overfitting, the model employs L1 and L2 regularization with values of 0.003 and 0.01, respectively, instead of dropout layers. These regularization techniques promote sparsity and control weight magnitudes, enhancing model generalization and robustness. Key ECG features, including mean heart rate, maximum and minimum heart rates, and heart rate variability, are calculated every 30 seconds using the NeuroKit2 Python package. Additionally, raw EEG data is preprocessed using a band-pass filter between 0.25 and 30 Hz.

Before implementing the model, synthetic ECG data was generated to simulate signals with defined characteristics. Using a NeuroKit2 device, synthetic data was created at a baseline heart rate of 75 BPM, and random noise was progressively added to evaluate the robustness of the feature extraction process. A feature generate algorithm was applied to this synthetic data, and heart rate and heart rate variability features were plotted as a function of the SNR ratio. Using K-Medoids clustering, two distinct clusters were identified, revealing that below an SNR of 4 dB, these key metrics became unreliable.

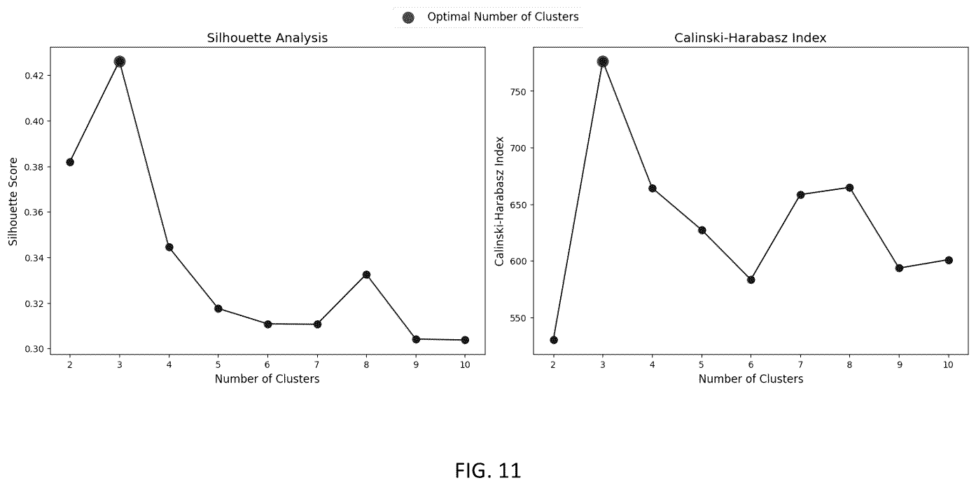

FIG. 11 shows graphs depicting the results of cluster analysis using K-Medoids algorithm, highlighting the evaluation of the optimal number of clusters. The left subplot presents the Silhouette Analysis, where the Silhouette Score is plotted against the number of clusters. The right subplot displays the Calinski-Harabasz Index, which evaluates clustering quality by comparing the variance between and within clusters. Both metrics confirm three as the optimal number of clusters.

The model implantation begins by calculating the SNR of the raw ECG signal over a duration of 30 seconds, with a 1-second shift, to ensure data quality. High-quality ECG data with an SNR greater than 4 dB was identified, and physiological features were extracted for further analysis. For workload predictions, features were exclusively derived from the high-quality data, and task-specific data was standardized relative to the baseline features. The standardized data was then utilized for workload predictions across various tasks. This systematic approach ensures that only reliable ECG data and features are used, significantly improving the accuracy and robustness of the workload predictions.

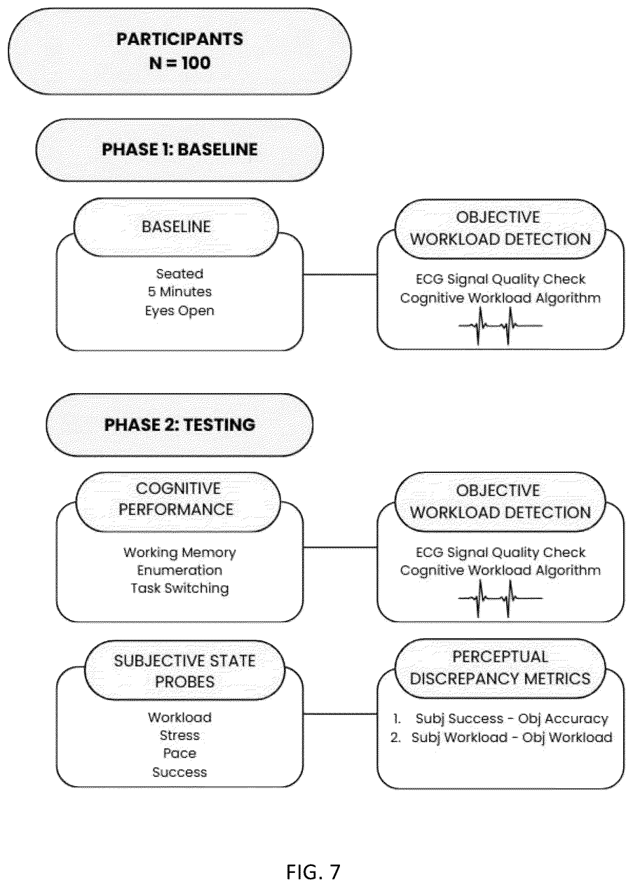

Participants were young adults (N=100; M Age=21.38, SD=3.27), racially diverse (37% Asian, 31% White/Caucasian, 21% Black/African, 8% Hispanic), and mostly male (76%) male. An overview of the study device and analysis pipeline is presented in FIG. 7, which illustrates a diagram depicting an overview of the phases of Baseline and Testing in accordance with embodiments of the present disclosure. Participants that were non-compliant with study instructions (did not do the tasks n=1, did not remain seated n=1), or appeared to fall asleep (n=3) during the testing were removed from analysis. The study protocol depicted in FIG. 7 shows all participants following phase 1 and 2 in the same session where objective workload (ECG data) was measured starting from baseline.

To analyze workload dynamics, baseline ECG statistics were computed for each participant and the entire dataset standardized based on these baseline values. The mean workload change from the baseline period and the mean accuracy for each tasks was calculated for each participant across nine data points (three cognitive tasks x three difficulty levels). These metrics were standardized across participants to facilitate meaningful comparisons. The analysis revealed the expected variations in accuracy such that as task difficulty increased accuracy decreased. This standardized approach served as a foundation for a workload classifier model, allowing for a personalized assessment of workload changes relative to each participant's baseline.

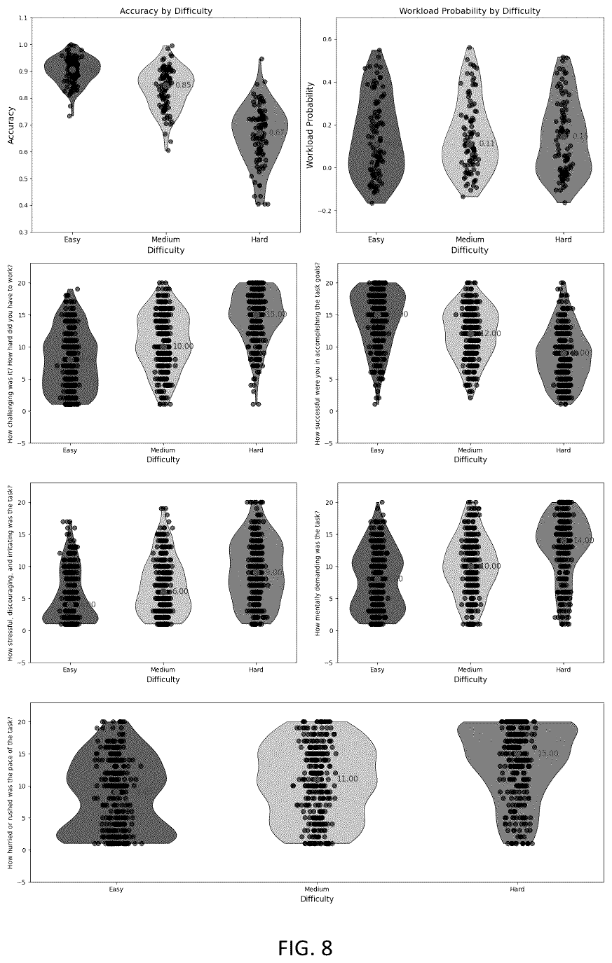

By focusing on workload changes relative to baseline, this approach addressed within-subject variability and captured unique individual characteristics and performance dynamics. It enabled the identification of patterns, trends, and outliers that may otherwise be obscured in aggregated data, providing a nuanced understanding of workload dynamics and a solid foundation for cluster analysis. FIG. 8 shows graphs depicting the variability among participants for task accuracy, workload, and each of the state probes. Referring to FIG. 8, the graphs show participant variability in accuracy and workload across different difficulty levels during the cognitive battery. The violin plots represent the distribution of accuracy (Top, left) and workload probability (Top, right) for Easy, Medium, and Hard tasks. Dots and labels indicate the median values for each difficulty level. Subsequent rows show violin plots for participant responses across difficulty levels (Easy, Medium, Hard) for each state probe (challenge, success, stress, mental demand, and perceived temporal demand. The violin plots display the distribution of responses with scatter points indicating individual responses and a median for each difficulty level. The final subplot is centered and maintains a consistent size with others for visual alignment.

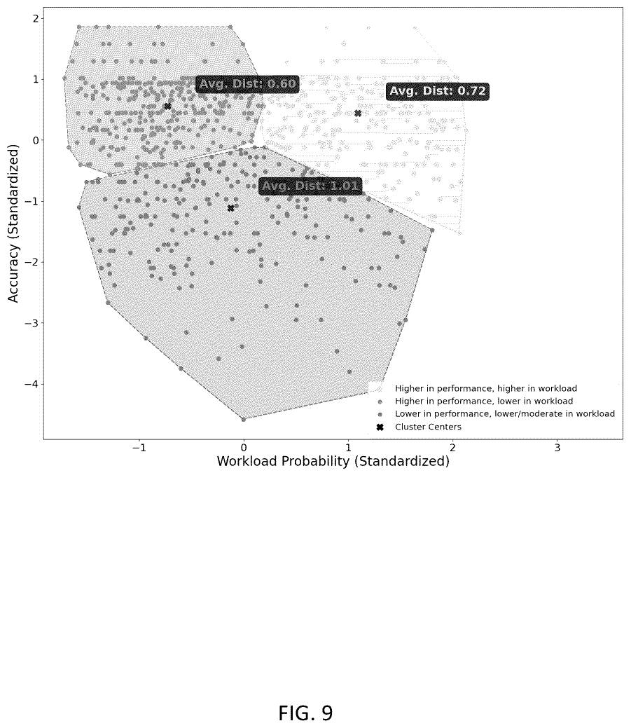

The K-Medoids were employed for clustering due to its robustness in handling noise and outliers. Unlike K-Means, K-Medoids identifies actual data points as cluster centers, providing more interpretable outcomes by minimizing dissimilarities rather than squared distances. FIG. 9 is a graph depicting standardized accuracy and workload metrics plotted with points colored according to their assigned clusters. Convex hulls outline each cluster's extent, and average distances to the cluster centers are labeled to indicate cohesion. Referring to FIG. 9, three clusters of workload probability and accuracy were identified using K-Medoids. The average distance from all points within each cluster to its centroid is indicated by text.

To determine the optimal number of clusters, the following were conducted:
1. Silhouette Analysis: Revealed three distinct clusters, with a high silhouette score indicating well-separated and cohesive clusters. The best silhouette score is 1, and the worst is −1.
2. Calinski-Harabasz Index: This metric evaluates clustering quality by comparing between-cluster and within-cluster variance. Higher scores indicate better clustering performance. Both analyses confirmed three clusters as optimal.

Centroid Proximity Index (CPI) measures the average distance of points within a cluster to its centroid, with smaller distances indicating stronger clusters. Among the three clusters, the one in the upper-left quadrant of FIG. 9, characterized by higher accuracy and lower workload, had the smallest CPI.

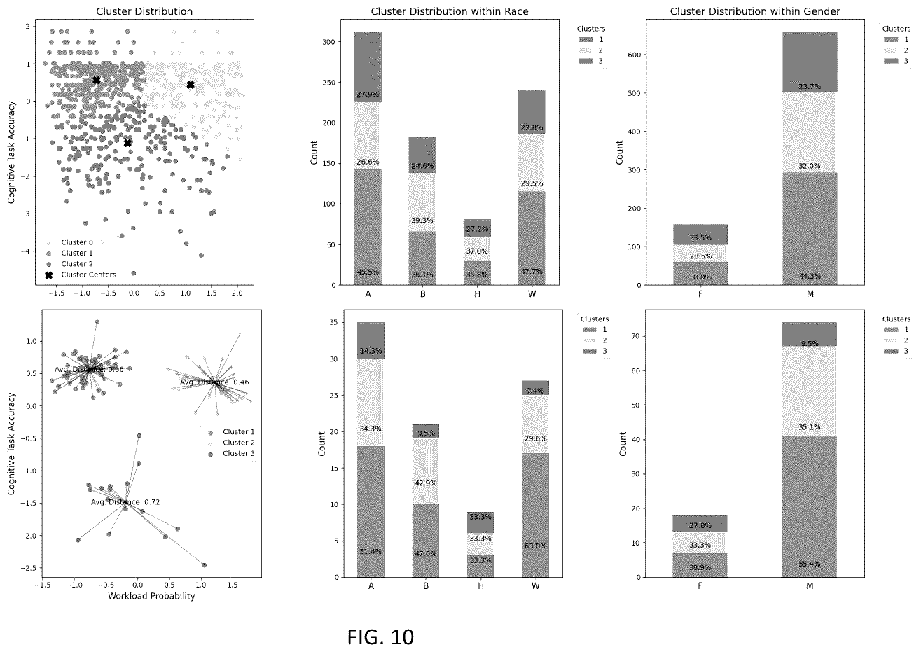

A goal of one study was to test the hypothesis that found distinct phenotypes would emerge characterized by high and low accuracy and high and low workload patterns across the tasks. Each participant's nine performances (three tasks x three difficulty levels) were plotted (FIG. 10). FIG. 10 are graphs depicting cluster analysis of all data points (3 tasks×3 difficulty levels). In the upper panel of FIG. 10, the left-most graph is a scatter plot of standardized workload probability and accuracy with clusters and centroids (indicated by black crosses). The middle graph of the upper panel in FIG. 10 shows cluster distribution by racial groups (W: White/Caucasian, A: Asian, B: Black/African-American, H: Hispanic/Latino). The right graph of the upper panel shows cluster distribution by gender (F: Female, M: Male). In the lower panel of FIG. 10, the graphs depict cluster analysis showing the same results using mode cluster membership across 9 data points per subject. Chi-square tests for proportional data were significant ps<0.05 for racial composition across cluster and for gender. More white and Asian individuals were represented in Cluster 1, and women are over represented in cluster 3. As can be seen, three, not four functionality distinct clusters emerge as indicated by the K Medoids algorithm: high accuracy: high workload, high accuracy: low workload, and low accuracy: low/moderate workload. A chi-square test of independence showed that gender (X² (2, 100)=11.31, p=<0.05) and race (X² (2, 100)=37.76, p=<0.05) varied significantly by cluster; Asian and White individuals characterize Cluster 1, black individuals are over represented in Cluster 2, and women are over represented in Cluster 3.

To test the hypothesis that Cluster 1 would who less discrepancy between subjective state probes and objective measures for success and mental demand than the other clusters, a 3-way ANOVA for each state probe of interested was conducted. For the analysis, three independent factors were test type (enumeration, working memory, task switching), difficulty level (easy, medium, hard), and modal cluster (1, 2, 3; See Table 1 below).

TABLE 1
ANOVA table - Three-factor ANOVAs were conducted for 3 discrepancy metrics; success,
mental demand, and temporal demand. For Success, there was a significant Task Level
Interaction. For Mental Demand, there was a significant Biotype Level interaction.

| | Subjective Success - | | | | | Sub Mental Demand - | | | | | Subjective Temporal Demand - | | | | |
|---|---|---|---|---|---|---|---|---|---|---|---|---|---|---|---|
| | Objective Accuracy | | | | | Objective Workload | | | | | Objective Workload | | | | |
| | df | F | n² | p | 95% CI | df | F | n² | p | 95% CI | df | F | n² | p | 95% CI |
| Biotype | 1 | [symbol] | [symbol] | [symbol] | [symbol] | 1 | [symbol] | [symbol] | [symbol] | [symbol] | 1 | [symbol] | [symbol] | [symbol] | [symbol] |
| Task | 2 | [symbol] | [symbol] | [symbol] | [symbol] | 2 | [symbol] | [symbol] | [symbol] | [symbol] | 2 | [symbol] | [symbol] | [symbol] | [symbol] |
| Level | 2 | [symbol] | [symbol] | [symbol] | [symbol] | 2 | [symbol] | [symbol] | [symbol] | [symbol] | 2 | [symbol] | [symbol] | [symbol] | [symbol] |
| Biotype [symbol] Task | 2 | [symbol] | | | [symbol] | 2 | [symbol] | | [symbol] | [symbol] | 2 | [symbol] | | [symbol] | [symbol] |
| Task [symbol] Level | 4 | [symbol] | [symbol] | [symbol] | [symbol] | 2 | [symbol] | | [symbol] | [symbol] | 2 | [symbol] | | [symbol] | [symbol] |
| Biotype [symbol] Level | 2 | [symbol] | | [symbol] | [symbol] | 4 | [symbol] | | [symbol] | [symbol] | 4 | [symbol] | | [symbol] | [symbol] |
| Biotype [symbol] Task [symbol] Level | 4 | [symbol] | | [symbol] | [symbol] | 4 | [symbol] | | [symbol] | [symbol] | 4 | [symbol] | | [symbol] | [symbol] |

[symbol]

indicates data missing or illegible when filed

Significant main effects for cluster, task, and level across all three subjective-objective comparisons were observed. For the comparison of Subjective Success and Objective Accuracy, Cluster 1 showed the highest discrepancy, which was negative, suggesting they thought they were less successful on the task than their performed showed. Cluster 2 showed less discrepancy than Cluster 1, and Cluster 3 showed almost no difference. For the comparison of Subject Mental Demand-Objective Workload and Subject Temporal Demand, the pattern was the opposite. Clusters 2 and 3 showed the highest levels of discrepancy on each task at each level whereas Cluster 1 showed the least.

This study investigated the relationship between subject and objective assessment of cognitive workload and performance during a set of cognitive tasks designed to elicit a range of physiological responses. The individual differences in cognitive performance and its associated workload, clustering participants based on performance and workload dynamics through an integrated clustering approach that combined cognitive performance accuracy with state probes and ECG-based workload measurements. The findings provide novel insights into cognitive task appraisal, physiological cost, and perceptual bias, offering both theoretical and practical implications for stress and workload assessment and research.

The results supported the hypothesis that distinct phenotypes emerge based on cognitive performance and physiological workload. Using K-Medoids clustering, three distinct clusters were identified, reflecting variability in accuracy and workload profiles across participants. These clusters demonstrate the value of integrating behavioral and physiological measurements as well as subjective and objective comparisons of the same phenomena in understanding cognitive task performance. For example, Cluster 1 participants exhibited high accuracy and low workload, aligning with a more efficient phenotype, whereas Cluster 3 participants displayed low accuracy and high workload, indicative of a potentially maladaptive response to cognitive challenges. Interestingly, the emergence of three clusters instead of the anticipated four suggests that individuals with below-average performance across tasks do not stratify into district subgroups based on workload (e.g., low performance with either high or low workload). This finding highlights the possibility that below-average performance might reflect a general difficulty in adapting to cognitive challenges, regardless of workload intensity.

Research on cognitive phenotyping has suggested that maladaptive responses may coalesce around factors like limited cognitive flexibility or resource strategies, which may explain the absence of further differentiation among low-performing individuals. This suggests that interventions targeting low performers may benefit from addressing broader cognitive and physiological adaptability rather than workload-specific patterns.

Additionally, the findings reveal significant objective-subjective discrepancies in workload appraisals across clusters. Specifically, participants in Cluster 1 reported more discrepancy in workload appraisals across clusters. Specifically, participants in Cluster 1 reported more discrepancy in workload appraisals across clusters. Specifically, participants in Cluster 1 reported more discrepancy between the subject state probe for task success and the objective accuracy and the differences was negative, indicating they underestimated their success. Clusters 2 and 3 did this as well but to a lesser extent. Conversely, participants in Clusters 2 and 3 exhibited what appears to be a negative perceptual bias, overestimating their workload relative to objective measures, which may signal increased cognitive strain, lower risk task efficiency, lower functional brain performance, and less fitness.

These findings contribute to a growing body of evidence on individual differences in performance under stress. The identification of functionally distinct phenotypes highlights the importance of personalized approaches to studying workload and task performance. By integrating ECG-based workload estimation with subject state probes, this study addresses the limitations of unitary measures of stress or performance, offering a more nuanced understanding of task appraisal dynamics. For example, a meta-analysis study examined cognitive flexibility in aging, highlighting that age-related reductions in cognitive flexibility were associated with both decreased and increased activation in several functional brain networks. This suggests that variability in cognitive flexibility may contribute to differences in task performance and workload management among individual and warrants further investigation.

The present study underscores the utility of physiological workload models, particularly ECG-based algorithms, in assessing cognitive demands. Compared to more invasive or logistically complex method such as EEG, ECG data can provide a practical and scalable approach to monitoring real-time cognitive load in various settings, including workplace, military, and education environments. In embodiments, this approach has application in designing personalized interventions to enhance performance and mitigate stress-related health risks over time. For example, a study identified a cognitive biotype of depression marked by treatment resistance, impaired cognitive control, and dysfunction in cognitive control brain circuits. The study showed that transcranial magnetic stimulation showed significant positive changes in connectivity within the cognitive control circuit specific to those with reduced connectivity at baseline while those with intact connectivity at baseline showed no significant changes. Cluster 3 performers here, characterized by low performance with a range of workloads align with some findings and warrant further investigation.

The relationship between, and cognitive workload and health outcomes has revealed both exaggerated and blunted physiological reactions were linked to adverse health effects, including psychopathology, cardiovascular morbidity and mortality. By characterizing how individuals respond to cognitive challenges at varying levels of difficulty, this study provides a foundation for understanding how workload-related phenotypes or biotypes may inform precision health interventions. For example, Cluster 3 participants may benefit from targeted strategies to improve task efficacy and reduce workload-related stress, whereas participants in Cluster 2 benefit mostly from workload reduction under challenge.

Conversely, Cluster 1 participants, who demonstrate efficient performance and accurate self-appraisal, may serve as a model for interventions aimed at enhancing resilience and adaptive task engagement. These findings align with prior research linking discrepant self-assessment with psychopathology and dysfunctional states suggesting a potential pathway for health promotion through improved cognitive workload management and awareness.

An important benefit of this study is the integration of subjective and objective measures, which provides a comprehensive framework for evaluating cognitive workload. The use of ECG-based workload estimation offers a practical and non-invasive approach to assessing physiological responses, while state probes capture subject perceptions of stress, challenge, and success. This dual approach can allow for a more nuanced understanding of the interplay between perceived and actual levels of workload.

In accordance with embodiments, systems are disclosed for assessing and improving mind-body health. These systems can evaluate mental performance under varying levels of difficulty that mimic real-world stress and measure how a person's body responds to that stress by analyzing the person's heart response. This information can be processed to determine the person's unique mind-body biotype. A report can be generated that summarizes this information and can provide personalized recommendations for improvement. As an example, the cognitive stress analyzer 110 as described herein can include these functionalities.

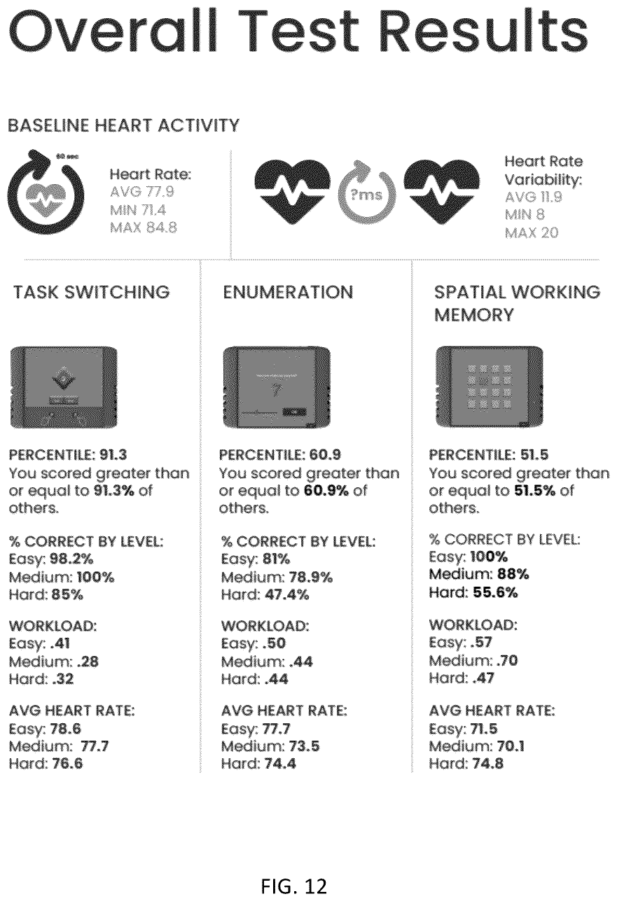

FIG. 12 is an example report of generated analysis information of a person about how the person's body responds to stress by analyzing the person's heart response. Referring to FIG. 12, the report includes baseline heart rate and heart rate variability information at a top portion. Further, the report presents task switching, enumeration, and spatial working memory estimated data in accordance with embodiments. Such information includes percentile information. Further, the report presents information about percentage correct, workload, and average heart rate by level (easy, medium, and hard). The user interface 108 can display the report on a display.

Task switching (TS) measures the flexibility of focused attention and requires shifting attention between competing goals. The anterior prefrontal cortex, at the front of the human brain, regulates this kind of executive function. When these skills are strong, individuals can better regulate their emotions, plan effectively, and maintain focus.

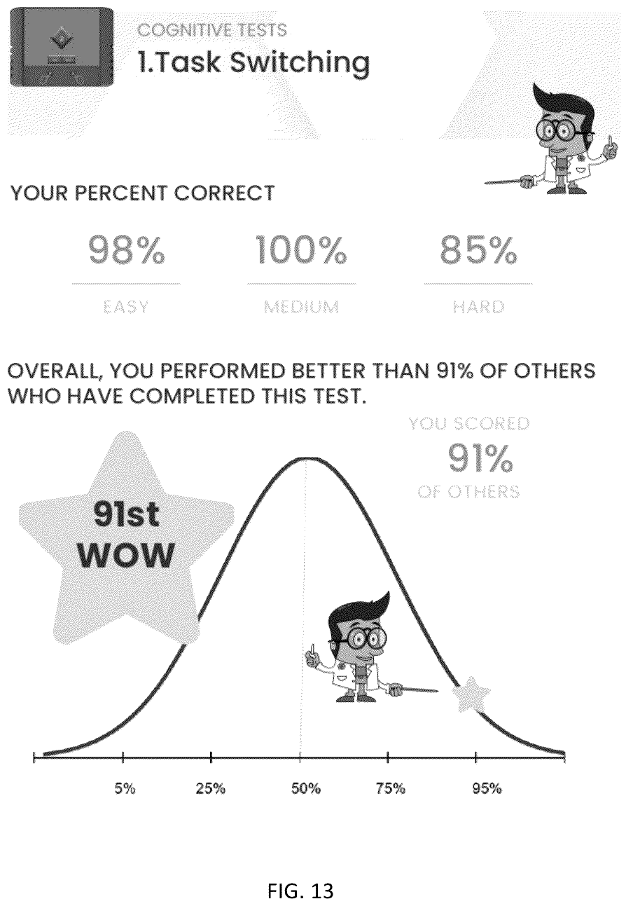

Task-switching performance, a measure of cognitive flexibility, is closely tied to both mental and physical health. Impairments in TS are observed in conditions like stress, depression, anxiety, aging and chronic diseases, while factors like regular exercise, quality sleep, and a nutrient-rich diet are associated with enhanced task-switching abilities and overall brain function. FIG. 13 is an example report of task switching analysis information. The user interface 108 can display the report on a display.

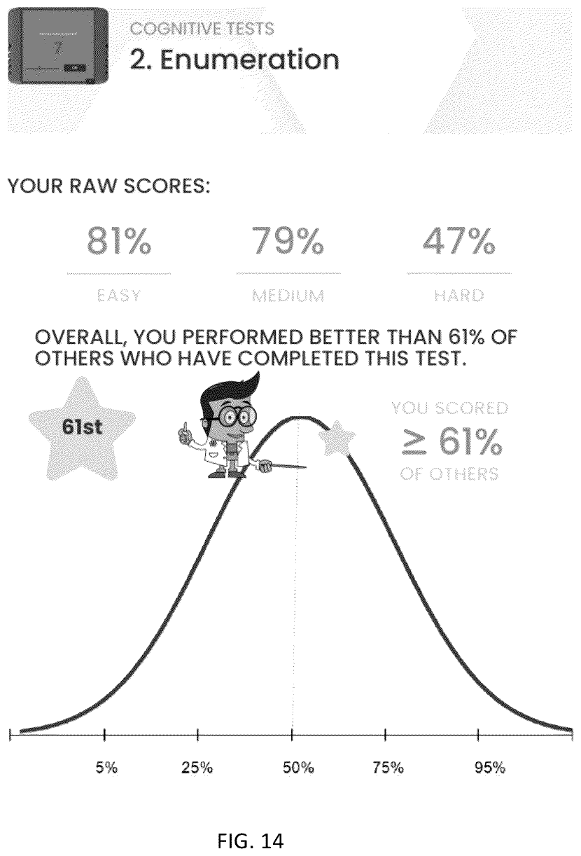

Enumeration (E) measures counting ability. Counting ability decreases with larger numbers of items (>5). Enumeration is governed by the prefrontal cortex, and other brain areas and challenges individuals with counting and remembering larger numbers of items in this range (5-9). The ability to hold larger amounts of information in order is related to cognitive health and is crucial for problem-solving, planning, and decision-making. These skills help maintain cognitive flexibility and adaptability, which are important for mental well-being. Being able to remember information under dynamic circumstances can reduce feelings of overwhelm and anxiety, leading to better mind-body balance. FIG. 14 is an example report of enumeration data. The user interface 108 can display the report on a display.

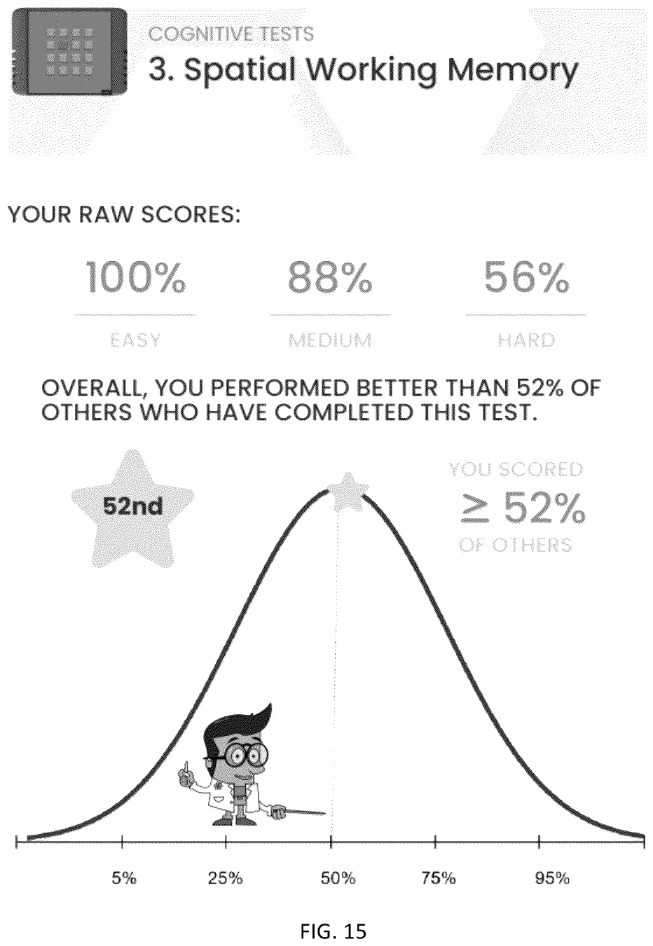

Spatial working memory (SWM) measures spatial short-term memory capacity. Performance decreases with increasing number of items remembered. These skills represent the “central executive” component of executive functioning which is sensitive to stress. The central executive may not store information itself but may direct resources to other components of the brain, making it essential for tasks requiring planning, problem-solving, and decision-making. Poor stress management and burnout can significantly impair SWM and reduce performance. SWM is regulated broadly by the neocortex. SWM can help with tasks that require planning, problem-solving, and navigation. Strong SWM can enhance cognitive flexibility, allowing people to adapt to new situations and solve problems more efficiently. FIG. 15 is an example report of spatial working memory data. The user interface 108 can display the report on a display.

Cognitive performance refers to the ability to process information accurately and efficiently across a variety of mental tasks. It can play a critical role in mind-body wellness, directly influencing how effectively individuals respond to challenges, make decisions, and maintain focus in both routine and high-pressure situations. Adults face an unprecedented barrage of cognitive demands requiring us to multitask as a way of life. These stressors can push our brains into overdrive, forcing us to retain information, switch focus quickly, and make rapid decisions, often at the expense of mental clarity and overall well-being.

Embodiments of the present disclosure can offer a window into your cognitive health by assessing three key abilities: Enumeration, Task Switching, and Spatial Working Memory. These scientifically validated tests can be leveraged by embodiments disclosed herein to evaluate distinct aspects of cognition at varying levels of difficulty (Easy, Medium, and Hard), providing a comprehensive picture of a person's mental agility and resilience. By understanding the person's cognitive performance, valuable insights can be presented into how the person's mind-body connection operates under different conditions, helping to identify areas for improvement and strategies for optimizing mental health and performance.

Heart rate is the number of times the heart beats in a minute. In general, a lower resting heart rate is healthier. The figure below is an example of a healthy resting heart rate, showing 60 beats in one minute. For most adults, regardless of gender or age, a normal resting heart rate is 60 to 100 beats per minute (bpm). The rate can be affected by a variety of health factors such as stress, anxiety, hormones, medication, and how physically active you are. People who are more active may have lower resting heart rates because their heart muscle does not need to work as hard. Studies have found that a higher resting rate is linked to lower physical fitness, higher blood pressure, and higher body weight. Heart rate may also be lower while sleeping.

Heart rate variability (HRV) is the difference in time between the beats and is measured in milliseconds. HRV indicates fitness of the autonomic nervous system and the ability to bounce back after stress. High HRV is often considered as an index of “health”. It is tied to risk and outcomes, so the more variation is healthier, as it shows your body's ability to adapt. The first figure below is an example of high/healthy HRV, showing a different time variation between each heart beat. The second figure shows low HRV, with no time variation between heart beats.

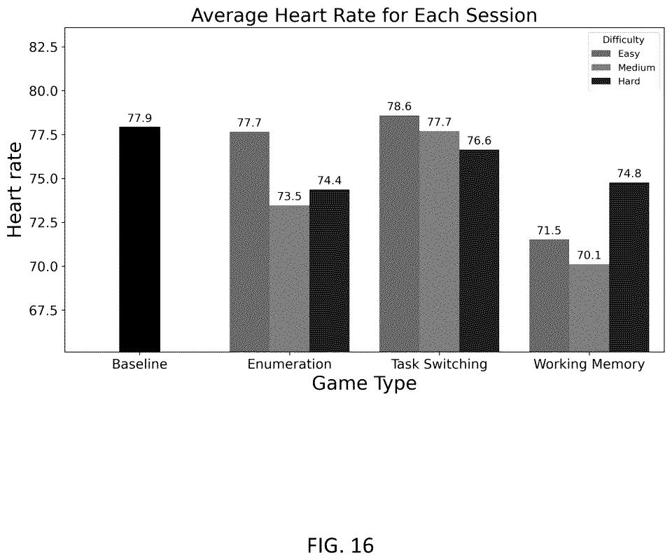

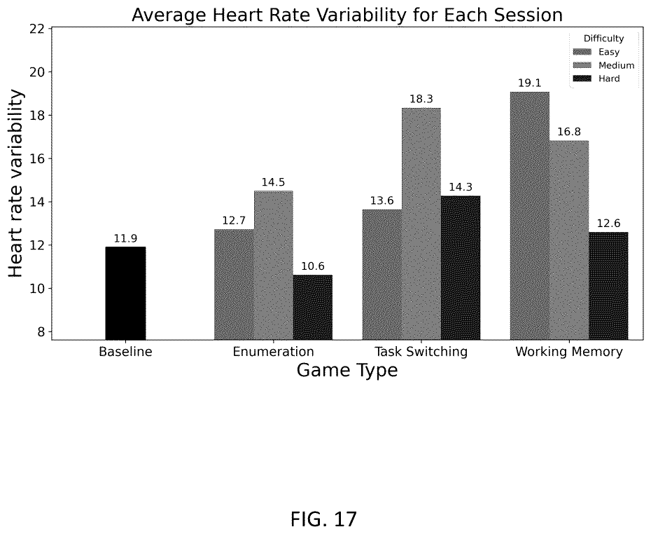

Heart rate and HRV reflect both long-term traits and short-term changes in your body. Factors like hydration, caffeine intake, sleep quality, and recent physical activity (e.g., walking or running upstairs) can affect these measurements. Since heart rate and HRV can vary from day to day. FIG. 16 is an example heart activity report. FIG. 17 is an example heart rate variability report. The user interface 108 can display the reports on a display.

In embodiments, heart activity can provide valuable insights into how a body responds to cognitive challenges. By measuring heart rate and HRV during a resting baseline and throughout each increasingly difficult block of tests, a detailed picture of physiological workload and adaptability under challenge can be generated, such as by the cognitive stress analyzer 110. The autonomic nervous system regulates many involuntary functions in the body, including heart rate, breathing, and digestion. It is divided into two branches: the parasympathetic nervous system, and the sympathetic nervous system. The parasympathetic nervous system (often referred to a “Rest and digest”) helps the body conserve energy, promotes relaxation, and supports restorative functions such as digestion and recovery. The sympathetic nervous system (often called “Fight or Flight”) prepares the body to respond to stress or danger by increasing heart rate, redirecting blood flow to muscles, and boosting alertness.

The interplay between these two branches creates HRV-the natural variation in the time between heartbeats. HRV reflects the balance and adaptability of the autonomic nervous system in responding to internal and external demands. High HRV is associated with a dominant parasympathetic response, indicating greater adaptability, improved recovery, better stress resilience, and enhanced cognitive performance. Low HRV reflects a dominant sympathetic response or reduced parasympathetic activity, often indicating chronic stress, fatigue, reduced adaptability, and impaired cognition.

Mental workload can be estimated by a machine learning algorithm, such as by cognitive stress analyzer 108, during the cognitive tests by beat-to-beat changes in heart rate. Workload is different from heart rate variability because this is how a person's body responds to mental effort. More resilient, healthy individuals usually have lower workloads during challenging times which is why they can feel better longer during stressful experiences. Other biotypes can have various consequences. For example, a low level of workload during cognitive tasks can be associated with feelings of boredom, disengagement, and loss of motivation. High workload can be associated with stress, burnout, mistakes, and poor performance. An optimal level of workload refers to that kind of “sweet spot” where tasks are challenging but manageable, leading to productivity and satisfaction.

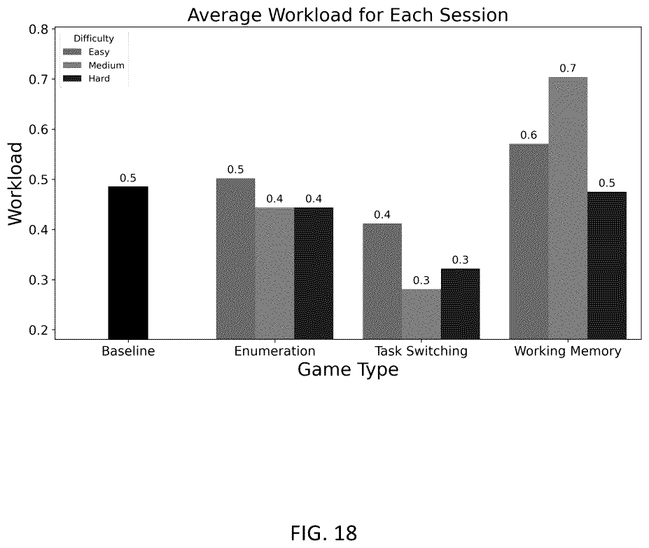

FIG. 18 is an example report showing average workload for each session. The user interface 108 can display the reports on a display. The graph depicts a person's workload across all testing phases. Workload is measured on a sale from 0 to 1, where higher values indicate greater mental effort. During the baseline (far left on the x-axis), your workload was about 0.5, meaning it was halfway to the maximum possible workload. The graph also shows a person's workload across three difficulty levels for each test: enumeration, task switching, and working memory. Workload was highest during the medium difficulty level of work memory. Workload was lowest during the medium difficulty level of task switching. It may be expected workload to be lowest during baseline and increase with task difficulty. However, individual patterns can vary. For instance, some people may experience elevated workload during the baseline phase due to nerves or adjusting to the testing setup.

Mental workload is the cognitive effort needed to process information, make decisions, and complete tasks. It is shaped by task difficulty, environment stressors, and an individual's skills and cognitive capacity.

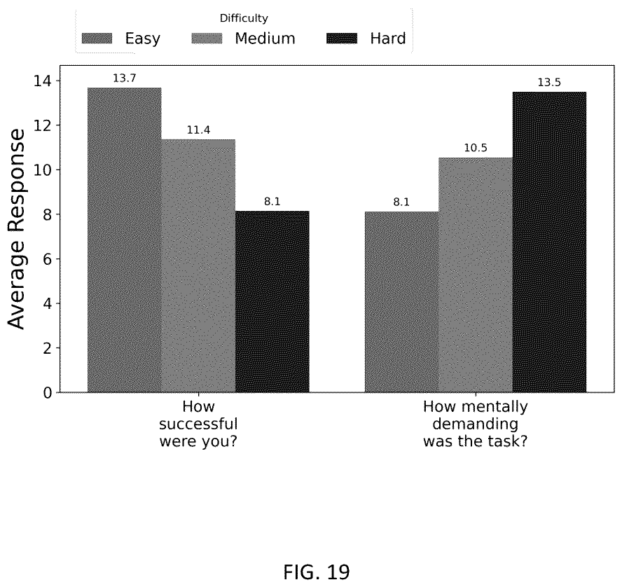

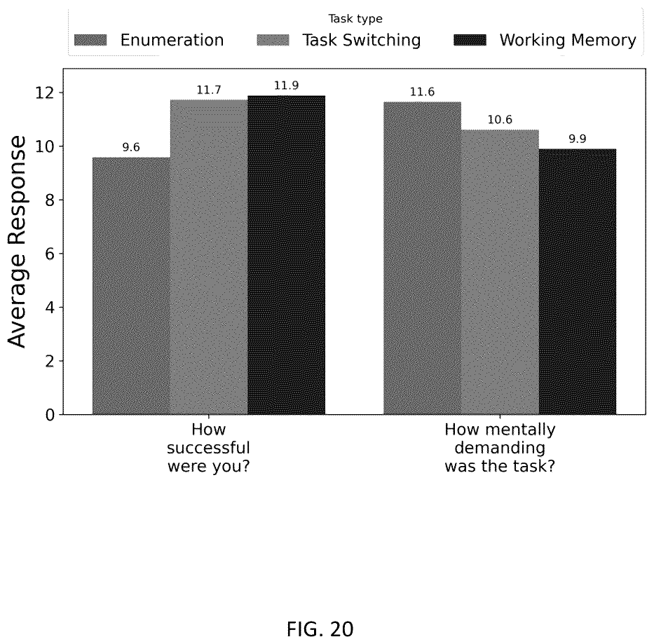

While moderate workload can enhance focus, excessive or prolonged demands may lead to cognitive fatigue, reduced performance, and stress. High mental workload also triggers physiological stress responses-like elevated heart rate and cortisol—which can contribute to chronic health issues such as hypertension and insomnia. FIG. 19 is a graph shows plots of how stressful and demanding a person rated difficulty levels across tasks. It can be seen that during testing it was noticed the increasing difficulty by reporting less success as it got harder. It was also reported that the person's mental demand increased with task difficulty. FIG. 20 is a graph showing average response as compared to input of “How successful were you?” and “How mentally demanding was the task?”.

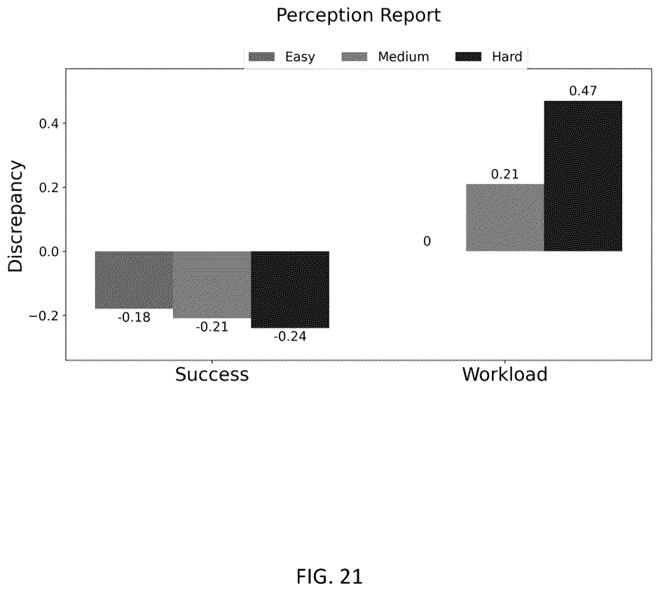

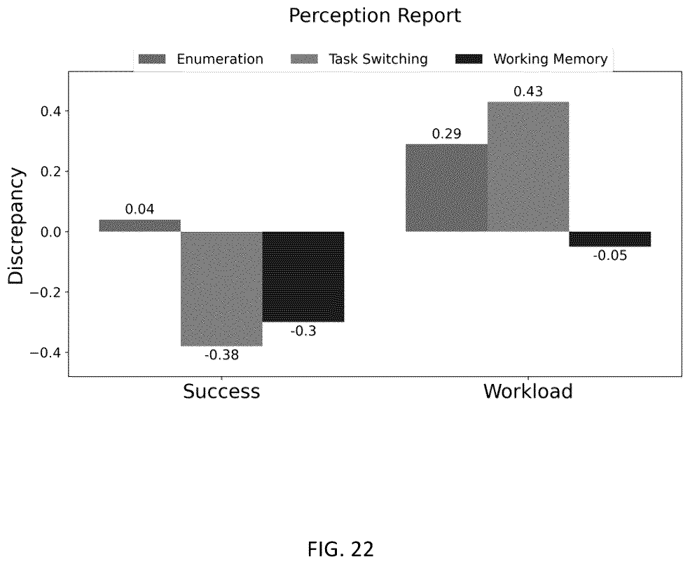

Calculated subjective-objective discrepancy was calculated by comparing a person's responses to the state probes with objective measures to see how accurately a person judged performance and workload during the tasks. This matters because discrepancies can go in different directions-some people overestimate their abilities or stress levels, or they may underestimate them, which can impact how you approach challenges. FIG. 21 is a graph depicting a plot of the difference between an estimation of task success and a person's actual accuracy for each level (left side). The estimation of workload and difference from the person's actual physiologic workload is shown on the right. Negative values reflect under-estimation, positive values indicate over-estimation. FIG. 22 is a graph depicting a plot of the difference between an estimation of task success and actual accuracy for each task (left side). The estimation of workload and difference from your actual physiologic workload is shown on the right. Negative values reflect under-estimation. Positive values indicate over-estimation.

A person's mind-body biotype reflects how the person's body and mind respond to mental stress and challenges. These patterns are generally stable but can change with the person's habits. Healthy routines, such as regular exercise, sleep, and stress management, can improve the person's biotype, while unhealthy habits may have the opposite effect. Knowing a person's biotype can be important because it can guide the person towards specific strategies to help with sustaining a healthy mind-body biotype or improve over time. Some biotypes may need to focus on building resilience through breathing techniques or by adopting consistent health routines, while others may focus on maintaining their already healthy system. By understanding the person's unique biotype, the person can make choices that help with managing stress better, performing well under pressure, and feel more confident.

The functional units described in this specification have been labeled as computing devices. A computing device may be implemented in programmable hardware devices such as processors, digital signal processors, central processing units, field programmable gate arrays, programmable array logic, programmable logic devices, cloud processing systems, or the like. The computing devices may also be implemented in software for execution by various types of processors. An identified device may include executable code and may, for instance, comprise one or more physical or logical blocks of computer instructions, which may, for instance, be organized as an object, procedure, function, or other construct. Nevertheless, the executable of an identified device need not be physically located together but may comprise disparate instructions stored in different locations which, when joined logically together, comprise the computing device and achieve the stated purpose of the computing device. In another example, a computing device may be a server or other computer located within a retail environment and communicatively connected to other computing devices (e.g., POS equipment or computers) for managing accounting, purchase transactions, and other processes within the retail environment. In another example, a computing device may be a mobile computing device such as, for example, but not limited to, a smart phone, a cell phone, a pager, a personal digital assistant (PDA), a mobile computer with a smart phone client, or the like. In another example, a computing device may be any type of wearable computer, such as a computer with a head-mounted display (HMD), or a smart watch or some other wearable smart device. Some of the computer sensing may be part of the fabric of the clothes the user is wearing. A computing device can also include any type of conventional computer, for example, a laptop computer or a tablet computer. A typical mobile computing device is a wireless data access-enabled device (e.g., an iPHONE® smart phone, an iPAD® device, smart watch, or the like) that is capable of sending and receiving data in a wireless manner using protocols like the Internet Protocol, or IP, and the wireless application protocol, or WAP. This allows users to access information via wireless devices, such as smart watches, smart phones, mobile phones, pagers, two-way radios, communicators, and the like. Wireless data access is supported by many wireless networks, including, but not limited to, Bluetooth, Near Field Communication, CDPD, CDMA, GSM, PDC, PHS, TDMA, FLEX, REFLEX, iDEN, TETRA, DECT, DataTAC, Mobitex, EDGE and other 2G, 3G, 4G, 5G, and LTE technologies, and it operates with many handheld device operating systems, such as EPOC, Windows CE, FLEXOS, OS/9, JavaOS, iOS and Android. Typically, these devices use graphical displays and can access the Internet (or other communications network) on so-called mini- or micro-browsers, which are web browsers with small file sizes that can accommodate the reduced memory constraints of wireless networks. In a representative embodiment, the mobile device is a cellular telephone or smart phone or smart watch that operates over GPRS (General Packet Radio Services), which is a data technology for GSM networks or operates over Near Field Communication e.g. Bluetooth. In addition to a conventional voice communication, a given mobile device can communicate with another such device via many different types of message transfer techniques, including Bluetooth, Near Field Communication, SMS (short message service), enhanced SMS (EMS), multi-media message (MMS), email WAP, paging, or other known or later-developed wireless data formats. Although many of the examples provided herein are implemented on smart phones, the examples may similarly be implemented on any suitable computing device, such as a computer.

An executable code of a computing device may be a single instruction, or many instructions, and may even be distributed over several different code segments, among different applications, and across several memory devices. Similarly, operational data may be identified and illustrated herein within the computing device, and may be embodied in any suitable form and organized within any suitable type of data structure. The operational data may be collected as a single data set, or may be distributed over different locations including over different storage devices, and may exist, at least partially, as electronic signals on a system or network.

The described features, structures, or characteristics may be combined in any suitable manner in one or more embodiments. In the following description, numerous specific details are provided, to provide a thorough understanding of embodiments of the disclosed subject matter. One skilled in the relevant art will recognize, however, that the disclosed subject matter can be practiced without one or more of the specific details, or with other methods, components, materials, etc. In other instances, well-known structures, materials, or operations are not shown or described in detail to avoid obscuring aspects of the disclosed subject matter.

As used herein, the term “memory” is generally a storage device of a computing device. Examples include, but are not limited to, read-only memory (ROM) and random access memory (RAM).

The device or system for performing one or more operations on a memory of a computing device may be a software, hardware, firmware, or combination of these. The device or the system is further intended to include or otherwise cover all software or computer programs capable of performing the various heretofore-disclosed determinations, calculations, or the like for the disclosed purposes. For example, exemplary embodiments are intended to cover all software or computer programs capable of enabling processors to implement the disclosed processes. Exemplary embodiments are also intended to cover any and all currently known, related art or later developed non-transitory recording or storage mediums (such as a CD-ROM, DVD-ROM, hard drive, RAM, ROM, floppy disc, magnetic tape cassette, etc.) that record or store such software or computer programs. Exemplary embodiments are further intended to cover such software, computer programs, systems and/or processes provided through any other currently known, related art, or later developed medium (such as transitory mediums, carrier waves, etc.), usable for implementing the exemplary operations disclosed below.

In accordance with the exemplary embodiments, the disclosed computer programs can be executed in many exemplary ways, such as an application that is resident in the memory of a device or as a hosted application that is being executed on a server and communicating with the device application or browser via a number of standard protocols, such as TCP/IP, HTTP, XML, SOAP, REST, JSON and other sufficient protocols. The disclosed computer programs can be written in exemplary programming languages that execute from memory on the device or from a hosted server, such as BASIC, COBOL, C, C++, Java, Pascal, or scripting languages such as JavaScript, Python, Ruby, PHP, Perl, or other suitable programming languages.

As referred to herein, the terms “computing device” and “entities” should be broadly construed and should be understood to be interchangeable. They may include any type of computing device, for example, a server, a desktop computer, a laptop computer, a smart phone, a cell phone, a pager, a personal digital assistant (PDA, e.g., with GPRS NIC), a mobile computer with a smartphone client, or the like.

As referred to herein, a user interface is generally a system by which users interact with a computing device. A user interface can include an input for allowing users to manipulate a computing device, and can include an output for allowing the system to present information and/or data, indicate the effects of the user's manipulation, etc. An example of a user interface on a computing device (e.g., a mobile device) includes a graphical user interface (GUI) that allows users to interact with programs in more ways than typing. A GUI typically can offer display objects, and visual indicators, as opposed to text-based interfaces, typed command labels or text navigation to represent information and actions available to a user. For example, an interface can be a display window or display object, which is selectable by a user of a mobile device for interaction. A user interface can include an input for allowing users to manipulate a computing device, and can include an output for allowing the computing device to present information and/or data, indicate the effects of the user's manipulation, etc. An example of a user interface on a computing device includes a GUI that allows users to interact with programs or applications in more ways than typing. A GUI typically can offer display objects, and visual indicators, as opposed to text-based interfaces, typed command labels or text navigation to represent information and actions available to a user. For example, a user interface can be a display window or display object, which is selectable by a user of a computing device for interaction. The display object can be displayed on a display screen of a computing device and can be selected by and interacted with by a user using the user interface. In an example, the display of the computing device can be a touch screen, which can display the display icon. The user can depress the area of the display screen where the display icon is displayed for selecting the display icon. In another example, the user can use any other suitable user interface of a computing device, such as a keypad, to select the display icon or display object. For example, the user can use a track ball or arrow keys for moving a cursor to highlight and select the display object.

The display object can be displayed on a display screen of a mobile device and can be selected by and interacted with by a user using the interface. In an example, the display of the mobile device can be a touch screen, which can display the display icon. The user can depress the area of the display screen at which the display icon is displayed for selecting the display icon. In another example, the user can use any other suitable interface of a mobile device, such as a keypad, to select the display icon or display object. For example, the user can use a track ball or times program instructions thereon for causing a processor to carry out aspects of the present disclosure.

As referred to herein, a computer network may be any group of computing systems, devices, or equipment that are linked together. Examples include, but are not limited to, local area networks (LANs) and wide area networks (WANs). A network may be categorized based on its design model, topology, or architecture. In an example, a network may be characterized as having a hierarchical internetworking model, which divides the network into three layers: access layer, distribution layer, and core layer. The access layer focuses on connecting client nodes, such as workstations to the network. The distribution layer manages routing, filtering, and quality-of-server (QoS) policies. The core layer can provide high-speed, highly-redundant forwarding services to move packets between distribution layer devices in different regions of the network. The core layer typically includes multiple routers and switches.

The present subject matter may be a system, a method, and/or a computer program product. The computer program product may include a computer readable storage medium (or media) having computer readable program instructions thereon for causing a processor to carry out aspects of the present subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flowchart illustrations and/or block diagrams of methods, apparatus (systems), and computer program products according to embodiments of the subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the embodiments have been described in connection with the various embodiments of the various figures, it is to be understood that other similar embodiments may be used, or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed is:

1. A system comprising:
an input interface configured to receive one or more acquired performance metrics that are indicative of a person's performance of a predetermined activity;
a cognitive stress analyzer configured to:
receive one or more acquired physiology metrics that are indicative of a condition of the person; and
analyze the one or more acquired physiology metrics and the one or more acquired performance metrics to generate an indicator of a mind-body fitness of the person;
a user interface configured to present the indicator of the mind-body fitness of the person.

2. The system of claim 1, wherein the one or more physiology metrics are acquired within a time period of the one or more performance metrics being acquired.

3. The system of claim 1, wherein the one or more acquired performance metrics include one of a firearm accuracy activity, cognitive testing, computing device use activity, race car driving, and eSports performance.

4. The system of claim 1, wherein the one or more acquired physiology metrics include one of heart rate, heart rate variability, blood pressure, oxygen saturation, respiratory rate, body temperature, blood glucose level, oxygen consumption, electrodermal activity, electroencephalography, pupillometry, gaze position and velocity, and muscle activation.

5. The system of claim 1, wherein the person is a first person, and
wherein the cognitive stress analyzer configured to:
receive indicators of mind-body fitness of a plurality of other people;
analyze the indicator of the mind-body fitness of the first person in comparison to the indicators of mind-body fitness of a plurality of other people; and
use the user interface to present the analysis.

6. The system of claim 1, wherein the cognitive stress analyzer is configured to compare a coefficient of variation of the one or more acquired physiology metrics and the one or more acquired performance metrics to generate an indicator of a mind-body fitness of the person.

7. The system of claim 1, wherein the cognitive stress analyzer is configured to acquire heart rate and heart rate variability during a resting baseline during different degrees of difficulty tests administered to the person, and
wherein the physiology metrics and the performance metrics are acquired during administration of the different degrees of difficulty tests.

8. A method comprising:
receiving one or more acquired performance metrics that are indicative of a person's performance of a predetermined activity;
receiving one or more acquired physiology metrics that are indicative of a condition of the person;
analyzing the one or more acquired physiology metrics and the one or more acquired performance metrics to generate an indicator of a mind-body fitness of the person; and
present, via a user interface, the indicator of a mind-body fitness of the person.

9. The system of claim 8, wherein the one or more physiology metrics are acquired within a time period of the one or more performance metrics being acquired.

10. The method of claim 8, wherein the one or more acquired performance metrics include one of a firearm accuracy activity, cognitive testing, computing device use activity, race car driving, and eSports performance.

11. The method of claim 8, wherein the one or more acquired physiology metrics include one of heart rate, heart rate variability, blood pressure, oxygen saturation, respiratory rate, body temperature, blood glucose level, oxygen consumption, electrodermal activity, electroencephalography, pupillometry, gaze position and velocity, and muscle activation.

12. The method of claim 8, wherein the person is a first person, and
wherein the method further comprises:
receiving indicators of mind-body fitness of a plurality of other people;
analyzing the indicator of the mind-body fitness of the first person in comparison to the indicators of mind-body fitness of a plurality of other people; and
using the user interface to present the analysis.

13. The method of claim 8, further comprising comparing a coefficient of variation of the one or more acquired physiology metrics and the one or more acquired performance metrics to generate an indicator of a mind-body fitness of the person.

14. The method of claim 8, further comprising acquiring heart rate and heart rate variability during a resting baseline during different degrees of difficulty tests administered to the person, and
wherein the physiology metrics and the performance metrics are acquired during administration of the different degrees of difficulty tests.

15. A computer program product comprising a computer readable storage medium having program instructions embodied therewith, the program instructions executable by a computing device to cause the computing device to:
receive one or more acquired performance metrics that are indicative of a person's performance of a predetermined activity;
receive one or more acquired physiology metrics that are indicative of a condition of the person;
analyze the one or more acquired physiology metrics and the one or more acquired performance metrics to generate an indicator of a mind-body fitness of the person; and
control a user interface to present the indicator of a mind-body fitness of the person.

16. The computer program product of claim 15, wherein the one or more physiology metrics are acquired within a time period of the one or more performance metrics being acquired.

17. The computer program product of claim 15, wherein the one or more acquired performance metrics include one of a firearm accuracy activity, cognitive testing, computing device use activity, race car driving, and eSports performance.

18. The computer program product of claim 15, wherein the one or more acquired physiology metrics include one of heart rate, heart rate variability, blood pressure, oxygen saturation, respiratory rate, body temperature, blood glucose level, oxygen consumption, electrodermal activity, electroencephalography, pupillometry, gaze position and velocity, and muscle activation.

19. The computer program product of claim 15, wherein the person is a first person, and
wherein the program instructions are executable by a computing device to cause the computing device to:
receive indicators of mind-body fitness of a plurality of other people;
analyze the indicator of the mind-body fitness of the first person in comparison to the indicators of mind-body fitness of a plurality of other people; and
use the user interface to present the analysis.

20. The computer program product of claim 15, wherein the program instructions are executable by a computing device to cause the computing device to compare a coefficient of variation of the one or more acquired physiology metrics and the one or more acquired performance metrics to generate an indicator of a mind-body fitness of the person.

21. The computer program product of claim 15, wherein the program instructions are executable by a computing device to cause the computing device to acquire heart rate and heart rate variability during a resting baseline during different degrees of difficulty tests administered to the person, and
wherein the physiology metrics and the performance metrics are acquired during administration of the different degrees of difficulty tests.
